SEQUENCE LISTING (7 sequences)

SEQ ID NO: 1 - protein, 4 aa, Artificial Sequence
  Peptide
REKA

SEQ ID NO: 2 - protein, 7 aa, Artificial Sequence
  Peptide
VHPKQHR

SEQ ID NO: 3 - protein, 17 aa, Artificial Sequence
  Peptide
NNQKIVNLKEKVAQLEA

SEQ ID NO: 4 - protein, 12 aa, Artificial Sequence
  Peptide
DITWDQLWDLMK

SEQ ID NO: 5 - protein, 5 aa, Artificial Sequence
  Peptide
CREKA

SEQ ID NO: 6 - protein, 9 aa, Artificial Sequence
  Peptide
CGVHPKQHR

SEQ ID NO: 7 - protein, 10 aa, Artificial Sequence
  Peptide
CGSPGWVRCG

(19) United States
(12) Patent Application Publication
(10) Pub. No.: US 2024/0050369 A1
(43) Pub. Date: Feb. 15, 2024

(54) Targeted Nanomedicine for Treating Vascular Disorders

(71) Applicants: THE UNIVERSITY OF CHICAGO, Chicago, IL (US); THE UNIVERSITY OF UTAH, Salt Lake City, UT (US)

(72) Inventors: Yun Fang, Chicago, IL (US); Matthew V. Tirrell, Chicago, IL (US); Yan-Ting Shiu, Salt Lake City, UT (US)

(21) Appl. No.: 17/766,318

(22) PCT Filed: Oct. 5, 2020
(86) PCT No.: PCT/US20/54291

§ 371 Date: Apr. 4, 2022

Related U.S. Application Data
(60) Provisional application No. 62/910,873, filed on Oct. 4, 2019.

(51) Int. Cl. A61K 9/107; A61K 47/10; A61K 47/18; A61K 47/42; A61K 45/06; A61K 31/7105; A61P 9/14

(52) U.S. Cl. CPC A61K 9/1075; A61K 47/10; A61K 47/183; A61K 47/42; A61K 45/06; A61K 31/7105; A61P 9/14

(57) ABSTRACT

This disclosure relates to compositions and methods for treating vascular disorders, including, for example, arteriovenous fistula (AVF) failure, stenosis, restenosis, and atherosclerosis.

### STATEMENT REGARDING FEDERALLY SPONSORED RESEARCH

This invention was made with government support under grant numbers R01HL138223 and R01DK100505 awarded by the National Institutes of Health (NIH). The government has certain rights in the invention.

### BACKGROUND OF THE DISCLOSURE

### Field of Invention

This disclosure relates to compositions and methods for treating vascular disorders, including, for example, arteriovenous fistula (AVF) failure, stenosis, restenosis, and atherosclerosis.

### Technical Background

Chronic kidney disease (CKD) affects approximately 37 million Americans and is defined as the gradual loss of kidney function. CKD can result from diabetes, high blood pressure, glomerulonephritis, polycystic kidney disease (PKD), and other disorders that lead to kidney failure over time. CKD increases the risk of cardiovascular disease and mortality as kidney function decreases. End-stage renal failure, also known as end-stage renal disease (ESRD; also known as end-stage kidney disease or ESKD), is the final, permanent stage of chronic kidney disease, where kidney function has declined to the point that the kidneys can no longer function on their own. There are >700,000 patients in the U.S. with ESRD. Most are treated with hemodialysis via the vascular access of arteriovenous fistula (AVF), a direct anastomosis between a peripheral artery and vein, which allows for higher blood flow (i.e., via a larger diameter) into and out of the dialyzer. However, AVF failure rate can be up to 60%, with costs for maintenance of vascular access averaging nearly $3 billion a year in the U.S. Moreover, there are currently no marketed drugs for treating AVF failure: anti-platelet agents (aspirins, dipyridamole . . . etc.), anti-coagulant drugs (Warfarin, heparin . . . etc.) and antihypertensive drugs (angiotensin receptor blockers, calcium channel blockers . . . etc.) have no clinically meaningful benefit in reducing AVF failure.

Atherosclerosis is a chronic inflammatory disease of the arterial wall that arises from an imbalanced lipid metabolism and a maladaptive inflammatory response, potentiated by the fluid mechanical stresses imposed on the endothelium. Atherosclerotic lesions are known to originate and develop preferentially at arterial sites of curvature, branches, and bifurcations, where complex hemodynamic conditions of disturbed blood flow are associated with chronically endoplasmic reticulum-stressed endothelial phenotypes expressing pro-inflammatory and pro-coagulant molecules. For example, the sinus of the carotid bifurcation with its recirculation is athero-susceptible whereas the nearby distal carotid artery, with straight streamlines, is athero-protected (FIG. 1). Treatment methodologies for atherosclerosis include, for example, percutaneous coronary intervention or coronary angioplasty when atherosclerotic plaques affect the coronary arteries. In the case of carotid blockage angioplasty and stenting can also be used.

The success of percutaneous coronary intervention has been dramatically improved by placement of coronary artery stents. In percutaneous coronary intervention, the stenosed coronary artery is dilated, and in stenting the mechanical support provided by the metal stent prevents elastic recoil. Still, hyperplasia forming a neointima within the stent leads to slow regrowth of the stenosis, referred to as in-stent restenosis. This has been addressed in many patients by using drug-eluting stents, for example releasing the mTOR inhibitor everolimus, which is now widely used. However, for patients who are particularly susceptible, such as diabetics, current therapy remains unsatisfactory. Moreover, usage of drug-eluting stent is associated with very late stent thrombosis (VLST), a potentially catastrophic complication.

MicroRNAs and Nanomedicine

MicroRNAs (miRNAs) are small non-coding RNA molecules, approximately 19-26 nucleotides in length, that regulate biological gene expression in diverse biological processes, miRNAs are conserved across organisms and regulate gene targets via hybridization to the 3′ untranslated region (UTR) of messenger RNA, thereby blocking the translation or degradation of mRNA targets. Altered miRNA expression is associated with multiple diseases and targeting of miRNA can be a treatment strategy in these cases.

Micelles are nanoparticles formed, for example, by self-assembly of amphiphilic block copolymers with a hydrophobic core that can serve as a reservoir for drug delivery. Micelles are small in size, allowing for penetration into tissues. They exhibit in vivo stability and are efficient in solubilizing water insoluble drugs, making them potentially useful therapeutic delivery tools. In this context, polyelectrolyte complexes forming micelles are the association complexes formed between oppositely charged particles (e.g., polymer-polymer, polymer-drug and polymer-drug-polymer).

Vascular diseases (including complications with AVF and athersclerosis interventions, as discussed above) although a leading cause of death worldwide, are significantly underserved by the nano-material community, especially relative to cancer nanomedicine which receives vastly more attention. One unique feature of vascular diseases is that pathological vascular remodeling, such as stenosis, typically occurs in specific sites of curvature, branching, and bifurcation where disturbed blood flows cause constitutive activation of vascular endothelium. For instance, disturbed flow-induced endothelial activation and vascular remodeling contribute to AVF failure in patients with ESRD undergoing AVF creation for hemodialysis. Targeted nanomedicine can augment future treatment of vascular diseases by suppressing endothelial activation and inhibiting stenosis “regionally” in diseased blood vessels.

### SUMMARY OF THE DISCLOSURE

This disclosure describes compositions and methods for treating vascular disorders, including, for example, arteriovenous fistula (AVF) failure, stenosis, restenosis, and atherosclerosis.

In a first aspect, the present disclosure provides a targeted nanoparticle, comprising an inhibitor of microRNA-92a (miR-92a). In some embodiments of the first aspect, the targeted nanoparticle comprises a polyelectrolyte micelle and a targeting molecule. In some embodiments, the polyelectrolyte micelle comprises a polyethylene glycol (PEG) domain and a domain of positively charged amino acids. In some embodiments, the PEG domain comprises PEG having an average molecular weight of about 1,000 to about 100,000 Daltons. In some embodiments, the domain of positively charged amino acids comprises repeats of lysine (K), arginine (R), and/or histidine (H). In some embodiments, the domain of positively charged amino acids comprises repeats comprising about 2 to about 100 residues. In some embodiments, the domain of positively charged amino acids comprises 30 repeats of lysine (K30). In some embodiments, the targeting molecule comprises a peptide comprising the amino acid sequence REKA (SEQ ID NO: 1), VHPKQHR (SEQ ID NO: 2), NNQKIVNLKEKVAQLEA (SEQ ID NO: 3), DITWDQLWDLMK (SEQ ID NO: 4), CREKA (SEQ ID NO: 5), CGVHPKQHR (SEQ ID NO: 6), or CGSPGWVRCG (SEQ ID NO: 7). In some embodiments, the targeted nanoparticle comprises VHPKQHR-PEG-K30, CGVHPKQHR-PEG-K30, NNQKIVNLKEKVAQLEA-PEG-K30, DITWDQLWDLMK-PEG-K30, REKA-PEG-K30, CREKA-PEG-K30, or CGSPGWVRCG-PEG-K30. In some embodiments, the miR-92a inhibitor comprises hsa-miR-92a-3p. In some embodiments, the miR-92a inhibitor comprises a concentration of about 2 μM.

In a second aspect, the present disclosure provides a pharmaceutical composition, comprising a therapeutically effective amount of a targeted nanoparticle comprising an miR-92a inhibitor and a pharmaceutically acceptable carrier, solvent, adjuvant, and/or diluent. In some embodiments of the second aspect, the pharmaceutical composition further includes a secondary therapeutic agent. In some embodiments, the secondary therapeutic agent comprises one or more of an anticoagulant, an antiplatelet agent, an angiotensin-converting enzyme inhibitor, an angiotensin II receptor blocker, an angiotensin-receptor neprilysin inhibitor, a beta blocker, a calcium channel blocker, a cholesterol-lowering medication, a digitalis preparation, a diuretic, a vasodilator, an anti-inflammatory medication, an IL-1b blocker, an inflammasome blocker, dehydroepiandrosterone sulfate, a myeloperoxidase inhibitor, a dipeptidyl peptidase-4 (DPP-4) inhibitor, a nitric oxide synthase activator, and/or a small GTPAse RhoA inhibitor. In some embodiments, the pharmaceutical composition is formulated for oral, intravenous, topical, ocular, buccal, systemic, nasal, injection, transdermal, rectal, or vaginal administration. In some embodiments, the pharmaceutical composition is formulated for inhalation or insufflation.

In a third aspect, the present disclosure provides a method of treating a vascular disorder in a subject, comprising administering to the subject a therapeutically effective amount of a pharmaceutical composition comprising a targeted nanoparticle comprising an miR-92a inhibitor. In some embodiments of the third aspect, the targeted nanoparticle comprises a polyelectrolyte micelle and a targeting molecule. In some embodiments of the third aspect, the polyelectrolyte micelle comprises a polyethylene glycol (PEG) domain and a domain of positively charged amino acids. In some embodiments of the third aspect, the PEG domain comprises PEG having an average molecular weight of about 1,000 to about 100,000 Daltons. In some embodiments of the third aspect, the domain of positively charged amino acids comprises repeats of lysine (K), arginine (R), and/or histidine (H). In some embodiments of the third aspect, the domain of positively charged amino acids comprises repeats comprising about 2 to about 100 residues. In some embodiments of the third aspect, the domain of positively charged amino acids comprises 30 repeats of lysine (K30). In some embodiments of the third aspect, the targeting molecule comprises a peptide comprising the amino acid sequence REKA (SEQ ID NO: 1), VHPKQHR (SEQ ID NO: 2), NNQKIVNLKEKVAQLEA (SEQ ID NO: 3), DITWDQLWDLMK (SEQ ID NO: 4), CREKA (SEQ ID NO: 5), CGVHPKQHR (SEQ ID NO: 6), or CGSPGWVRCG (SEQ ID NO: 7). In some embodiments of the third aspect, the targeted nanoparticle comprises VHPKQHR-PEG-K30, CGVHPKQHR-PEG-K30, NNQKIVNLKEKVAQLEA-PEG-K30, DITWDQLWDLMK-PEG-K30, REKA-PEG-K30, CREKA-PEG-K30, or CGSPGWVRCG-PEG-K30. In some embodiments of the third aspect, the miR-92a inhibitor comprises hsa-miR-92a-3p. In some embodiments of the third aspect, the miR-92a inhibitor comprises a concentration of about 2 μM.

In a fourth aspect, the present disclosure provides a method of treating a vascular disorder in a subject, comprising administering to the subject a therapeutically effective amount of a pharmaceutical composition comprising a targeted nanoparticle comprising an miR-92a inhibitor, wherein the targeted nanoparticle is preferentially targeted to inflamed endothelial cells associated with the vascular disorder and reducing inflammation at the site of the inflamed endothelial cells. In some embodiments of the fourth aspect, the vascular disorder comprises one or more of arteriovenous fistula (AVF) failure, stenosis, restenosis, and atherosclerosis. In some embodiments of the fourth aspect, the method results in one or more of greater lumen cross-sectional area, greater lumen diameter, or increased flow rate compared to a control at the site of the inflamed endothelial cells.

In a fifth aspect, the present disclosure provides a method of promoting endothelial wound healing in a subject comprising administering to the subject a therapeutically effective amount of a pharmaceutical composition comprising a targeted nanoparticle comprising an miR-92a inhibitor, wherein the targeted nanoparticle is preferentially targeted to inflamed endothelial cells associated with the endothelial wound and reducing inflammation at the site of the inflamed endothelial cells. In some embodiments of the fifth aspect, the method further includes stimulating endothelial growth at the site of the wound.

These and other features and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The accompanying drawings are included to provide a further understanding of the methods and compositions of the disclosure, and are incorporated in and constitute a part of this specification. The drawings illustrate one or more embodiment(s) of the disclosure, and together with the description serve to explain the principles and operation of the disclosure.

FIG. 1. Human carotid bifurcations with carotid sinus with recirculation (arrow), which is athero-susceptible. Other portions of the bifurcation experience linear laminar flow, which is athero-protective.

FIG. 2. Brachiocephalic fistula for hemodialysis. The cephalic arch and arteriovenous anastomosis where endothelial cells are constantly activated by locally disturbed flow are sites most susceptible to stenosis and thrombosis, leading to arteriovenous fistula (AVF) failure.

FIG. 3. AVF maturation requires limited inward remodeling and sufficient outward remodeling.

FIGS. 4A-4B. Flow at atherosclerosis-prone carotid artery bifurcation. (4A) Aberrant blood flow; (4B) upregulated microRNA-92a (miR-92a) in cultured endothelial cells (ECs) as shown by the ratio of miR-92a/U6, miRNA RT-PCR (Wu et a. Circulation 2011). PS: pulsatile shear flow (linear laminar flow); OS: oscillatory shear flow (disturbed flow).

FIGS. 5A-5B. Disturbed flow in human (5A) brachiocephalic arteriovenous anastomosis and (5B) cephalic arch after creation of arteriovenous fistula (AVF), which is similar to disturbed flow at atherosclerosis-prone carotid artery bifurcation (FIG. 4A).

FIG. 6. miR-92a down-regulates Sirtuin 1 (SIRT1), Krüppel-like factor 2 (KLF2), Krüppel-like factor 4 (KLF4), and endothelial nitric oxide synthase (eNOS), which leads to decreased flow-mediated dilation (FMD).

FIG. 7. Association of preoperative FMD with 6-week AVF diameter and blood flow. Preoperative FMD was positively associated with AVF maturation (Allon et al. JASN, 2016).

FIG. 8. Serum miR-92a levels were negatively associated with FMD in patients with coronary artery disease (Chen et al. Circulation 2015).

FIGS. 9A-9B. Relative serum miR-92a levels were increased in chronic kidney disease (CKD) patients (9A) and negatively associated with FMD (9B, Shang et al. JASN, 2017).

FIGS. 10A-10B. Relative CKD miR-92a serum levels. (10A) CKD upregulated relative miR-92a serum levels compared to control; (10B) normalized mRNA expression of endothelial protective genes in CKD serum-treated cultured ECs was observed with decreased miR-92a levels by miR-92a inhibitors (anti-miR92a) (Shang et al. JASN, 2017).

FIG. 11. Hypothetical role for miR-92a in AVF maturation: miR-92a leads to decreased expression of endothelial protective genes which causes increased inward AVF remodeling and decreased outward AVF remodeling, which contribute to AVF failure.

FIG. 12. Working hypothesis: a combination of CKD and AVF blood flow increases miR-92a expression causing AVF maturation failure.

FIG. 13. Study design for rat study.

FIG. 14. Modified, low-dose dietary adenine-induced CKD and AVF creation timeline, as described in Langer et al. Kidney International 2010.

FIG. 15. CKD rats have elevated serum miR-92a levels (Shang et al., JASN 2017), as seen in CKD patients.

FIG. 16. Study design for mouse study.

FIG. 17. Schematic of proposed effect of miR-92a inhibition on AVF development using whole-body gene knockout.

FIGS. 18A-18B. Mouse carotid-jugular AVFs were created in miR-92a knockout (KO) and wild-type (WT) C57BL/6 mice, as described in Chun el al. J Vis Exp 2016. MiR-92a KO decreased NH in AVF veins. (18A) Percent area of stenosis by NH (top) and Percent area of open lumen (bottom); (18B) Outward-to-Inward remodeling ratio.

FIG. 19. Schematic of proposed effect of miR-92a inhibition of AVF development using targeted nanomedicine.

FIGS. 20A-20B. Dual-function nanoparticles (NPs) encapsulate miR inhibitor and target inflamed endothelial cells. Vascular cell adhesion molecule (VCAM-1) presence in CKD rat tissue (20A) Non-surgical femoral artery; (20B) AVF artery. Dark color=VCAM1; arrows indicate endothelial cells.

FIG. 21. Dual-function NPs encapsulate miR inhibitor and target inflamed endothelial cells. Assembly and structure of VCAM-1 targeting nanoparticles with miR inhibitors in the core. The peptide targeting VCAM1 has the sequence VHPKQHR (SEQ ID NO: 2).

FIG. 22. Negatively stained TEM image of micelles formed via complexation of REKA-PEG-K30 and miR-92a inhibitor. PEG is polyethylene glycol.

FIG. 23. Preferential accumulation of VCAM-1-targeted mIR-92a inhibitors to aorta in vivo.

FIGS. 24A-24C. Comparison of control, naked and NP-encapsulated miR-92a inhibitor in treating neointimal hyperplasia. Nanoparticle-encapsulated miR inhibitors enhance AVF remodeling when compared to controls and naked inhibitors via a reduction in venous neointimal hyperplasia and promoted venous lumen expansion. (24A) morphometric analysis of open lumen area; (24B) Percent area of open lumen; and (24C) percent area of stenosis by neointimal hyperplasia (bottom). PBS (filled circle), naked inhibitors (open square) and nanoparticle-encapsulated inhibitors (shaded triangle).

FIG. 25. Study design for Bioinformatics study.

FIGS. 26A-26C. Bioinformatics study. (26A) Enrichment analysis suggested pathway differences between AVFs in miR-92a KO vs. WT mice; (26B) Network analysis suggested interactions among gene group I and other genes; (26C) Gene group I is associated with the extracellular matrix and protease activity.

FIGS. 27A-27C. Apo E^{-/-} mice (27A) were fed a high fat diet at week sixteen. At eighteen weeks, a tail vein injection was performed of either naked miR-92a inhibitor or the miR-92a inhibitor encapsulated in a VCAM1-targeted micellar nanoparticle. In both cases, a dose of 8 mg/kg or 4 mg/kg of body weight was administered. At twenty weeks, the mice were euthanized and the size (area) of atherosclerotic lesions in the aortic root was measured. Data from injections of a dose of 8 mg/kg are shown in 27B. Data from injections of a dose of 4 mg/kg are shown in 27C. 1.0 on the normalized vertical axis is the average size of the lesions in the mice that received injections of phosphate buffered saline (PBS) shown in column 1. Column 2 shows results for injection of the naked inhibitor. Column 3 shows the results for injection of the micellar nanoparticle-delivered control oligonucleotide of the same nucleic acid composition in a scrambled sequence targeted to tissue displaying VCAM1, an indication of local inflammation. Column 4 shows the results for the injection of the micellar nanoparticle-delivered miR-92a inhibitor targeted to tissue displaying VCAM1.

FIGS. 28A-28C. (28A) 16-week old Apo E^{-/-} mice were fed a high fat diet and subjected to carotid partial ligation in the left carotid. This surgical procedure (Nam et al. A. J. Phys. Heart, 2009) introduces disturbed blood flow and induces stenosis in left carotid artery in 14 days. (28B) Three days after the partial carotid ligation, a tail vein injection was conducted of either naked miR-92a inhibitor or the miR-92a inhibitor encapsulated in a VCAM1-targeted micellar nanoparticle. In both cases, a dose of 2 mg/kg of body weight was administered. Two more injections were conducted on days 6 and 9 after the partial carotid ligation. Mice were sacrificed 14 days after the partial carotid ligation to measure the stenosis in the partially-ligated left carotid arteries. (28C) Vertical axis is the average size of the stenosis in the partially-ligated carotid artery from the mice that received injections of phosphate buffered saline (PBS) shown in column 1. Column 2 shows the results for injection of the naked miR-92a inhibitor. Column 3 shows the results for injection of the micellar nanoparticle-delivered control oligonucleotide of the same nucleic acid composition in a scrambled sequence targeted to tissue displaying VCAM1, an indication of local inflammation. Column 4 shows the results for the injection of the micellar nanoparticle-delivered miR-92a inhibitor targeted to tissue displaying VCAM1. Representational cross-sections of each treatment group are shown below the data chart.

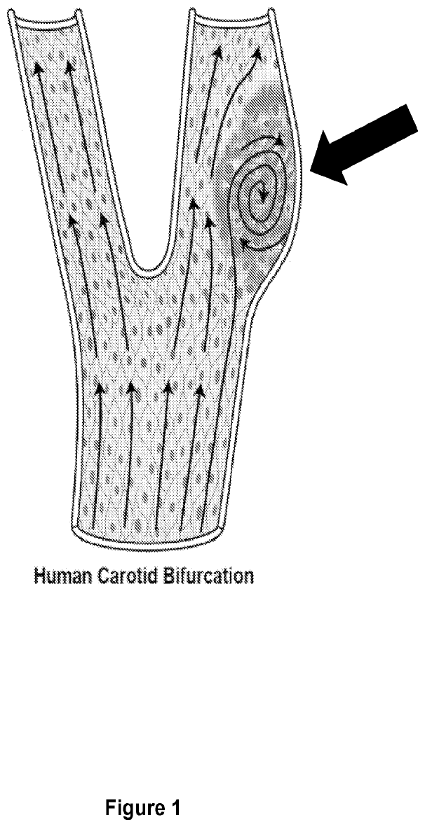

### DETAILED DESCRIPTION

Provided herein are compositions and methods for treating vascular disorders, including, for example, arteriovenous fistula (AVF) failure, stenosis, restenosis, and atherosclerosis. As used herein, the term “vascular disorder” refers to disorders, diseases, and/or damage to the vascular system of an individual. The vascular system, also known as the circulatory system, includes the vessels (e.g., arteries, veins, capillaries, and lymph vessels) that carry blood and lymphatic fluid throughout the body.

It is to be understood that the particular aspects of the specification are described herein are not limited to specific embodiments presented, and can vary. It also will be understood that the terminology used herein is for the purpose of describing particular aspects only and, unless specifically defined herein, is not intended to be limiting. Moreover, particular embodiments disclosed herein can be combined with other embodiments disclosed herein, as would be recognized by a skilled person, without limitation.

Throughout this specification, unless the context specifically indicates otherwise, the terms “comprise” and “include” and variations thereof (e.g., “comprises,” “comprising,” “includes,” and “including”) will be understood to indicate the inclusion of a stated component, feature, element, or step or group of components, features, elements or steps but not the exclusion of any other component, feature, element, or step or group of components, features, elements, or steps. Any of the terms “comprising,” “consisting essentially of,” and “consisting of” may be replaced with either of the other two terms, while retaining their ordinary meanings.

As used herein, the singular forms “a,” “an,” and “the” include plural referents unless the context clearly indicates otherwise.

In some embodiments, percentages disclosed herein can vary in amount by ±10, 20, or 30% from values disclosed and remain within the scope of the contemplated disclosure.

Unless otherwise indicated or otherwise evident from the context and understanding of one of ordinary skill in the art, values herein that are expressed as ranges can assume any specific value or sub-range within the stated ranges in different embodiments of the disclosure, to the tenth of the unit of the lower limit of the range, unless the context clearly dictates otherwise.

As used herein, ranges and amounts can be expressed as “about” a particular value or range. About also includes the exact amount. For example, “about 5%” means “about 5%” and also “5%.” The term “about” can also refer to ±10% of a given value or range of values. Therefore, about 5% also means 4.5%-5.5%, for example.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

“Pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problems or complications commensurate with a reasonable benefit/risk ratio or which have otherwise been approved by the United States Food and Drug Administration as being acceptable for use in humans or domestic animals.

“Therapeutically effective amount” or “effective amount” refers to that amount of a therapeutic agent, such as an miR-92A inhibitor, which when administered to a subject, is sufficient to effect treatment (e.g., improve symptoms) for a disease or disorder described herein, such as, for example, AVF failure, stenosis, restenosis, or atherosclerosis. The amount of a compound which constitutes a “therapeutically effective amount” or “effective amount” can vary depending on the compound, the disorder and its severity, and the age, weight, sex, and genetic background of the subject to be treated, but can be determined by one of ordinary skill in the art.

“Treating” or “treatment” as used herein refers to the treatment of a disease or disorder described herein, in a subject, preferably a human, and includes inhibiting, relieving, ameliorating, or slowing progression of the disease or disorder or one or more symptoms of the disease or disorder.

“Subject” refers to a warm blooded animal such as a mammal, preferably a human, which is afflicted with, or has the potential to be afflicted with one or more diseases and disorders described herein.

“Pharmaceutical composition” as used herein refers to a composition that includes one or more therapeutic agents disclosed herein, such as an miR-92A inhibitor, a pharmaceutically acceptable carrier, a solvent, an adjuvant, and/or a diluent, or any combination thereof.

As used herein, the term “vascular disease” can also be described as a “vascular disorder.”

In view of the present disclosure, the methods and compositions described herein can be configured by the person of ordinary skill in the art to meet the desired need. In general, the disclosed materials and methods provide improvements in treating vascular disorders as described herein.

### Overview

Cardiovascular diseases, undesired vascular remodeling, and obstruction of blood vessels are potentiated by disturbed blood flow such as vortex formation and other manifestations of local turbulence and flow complexity, interacting with other factors such as unbalanced lipid metabolism and uremia. Disturbed blood flow upregulates the production of a microRNA, miR-92a, which increases local inflammation, and promotes local, pathological vascular remodeling and stenosis. Disclosed herein are engineered nanoparticles that can locally deliver an inhibitor against miR-92a that retards the development of localized atherosclerotic lesions in ApoE knockout mice. The nanoparticles also inhibit pathological vascular remodeling in arteriovenous fistulae (AVF) in mice. It is further contemplated that the inflammation targeting nanoparticles carrying an miR-92a inhibitor are also effective at treating and/or preventing restenosis, which often occurs after insertion of a stent to relieve coronary artery blockage, for example, and after angioplasty.

Moreover, the present disclosure demonstrates that this targeted nanoparticle significantly reduced vascular stenosis, which is a narrowing of the arterial lumen that disrupts local blood flow and leads to a wide range of vascular diseases in the brain, bean, and legs.

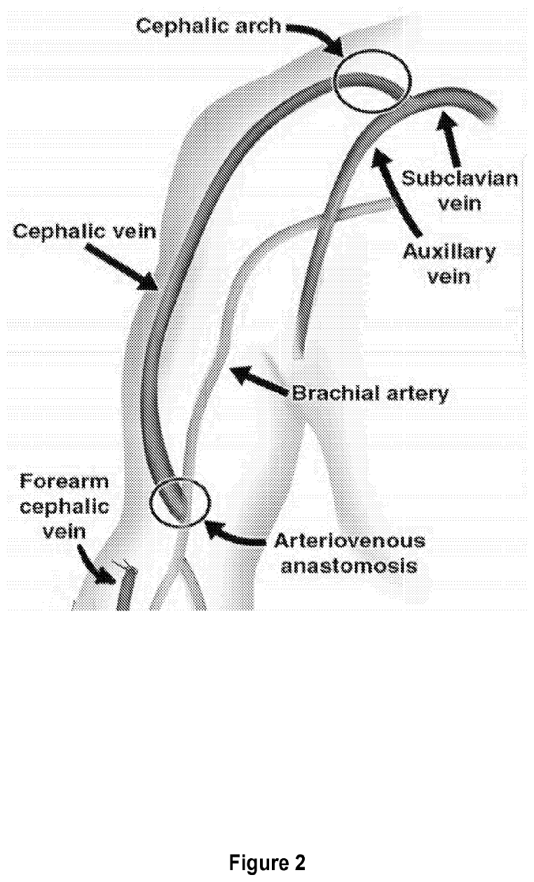

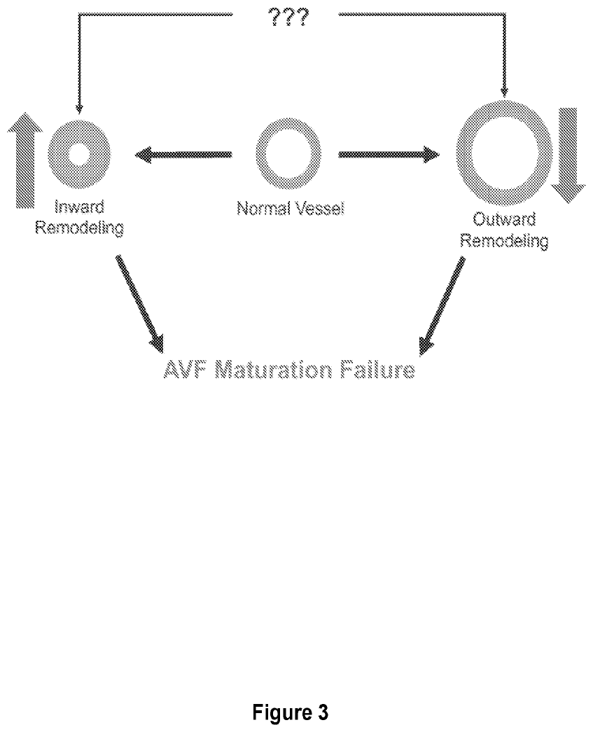

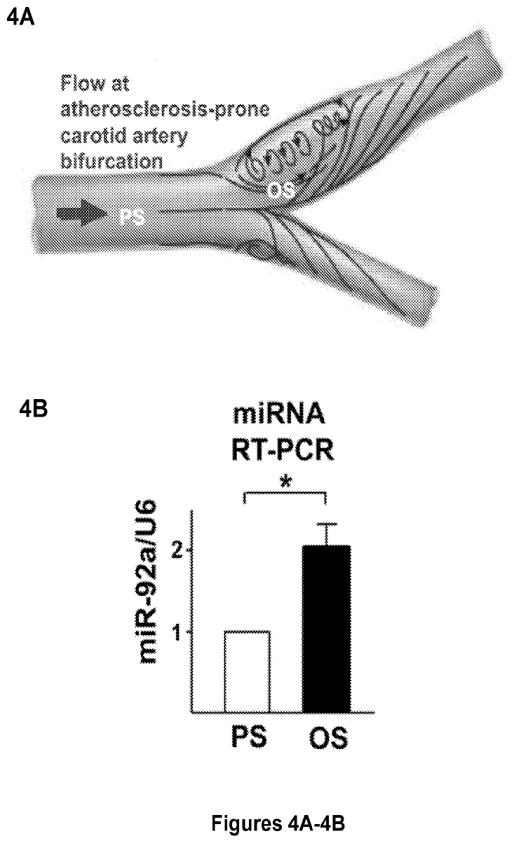

The preferred vascular access for hemodialysis is the arteriovenous fistula (AVF) that is surgically created by a direct anastomosis between a peripheral artery and vein (FIG. 2). Many AVFs fail to mature sufficiently for adequate dialysis due to vascular stenosis (FIG. 3). The failure rate of newly created AVFs varies from 25-60% N with the most common causes of AVF failure being pathological vascular remodeling, insult to the endothelial layer, stenosis, and thrombosis. The cephalic arch where endothelial cells are constantly activated by locally disturbed flow is one of the sites most susceptible to stenosis and thrombosis, leading to AVF failure. The annual cost of treating vascular access dysfunction totals over one billion dollars in the United States of America, largely due to new AVF creation, interventional procedure with angioplasty and stent placement, as well as complications from prolonged catheter use. There is currently no pharmacological treatment for AVF failure, representing a significant unmet clinical need.

MicroRNAs are small non-coding RNA molecules known to regulate pathological processes related to endothelial cell function and cardiovascular health. MicroRNAs (miRs), including miR-92a, are thought to play a role in arteriovenous fistula maturation. (FIGS. 2, 3, 6 and 12). Studies have shown that serum miR-92a levels were negatively associated with FMD in patients with coronary artery disease (Chen el al. Circulation 2015). Studies have also shown that preoperative FMD was positively associated with AVF maturation (Allon et al. JASN, 2016).

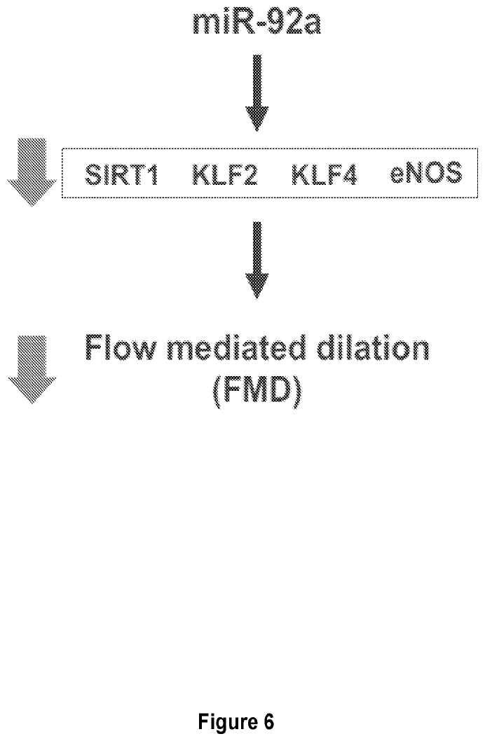

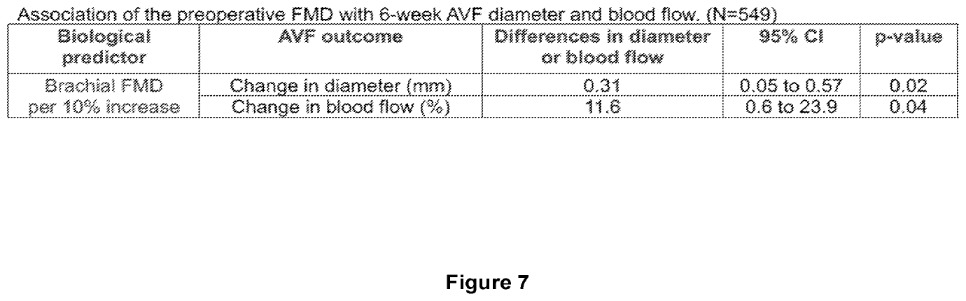

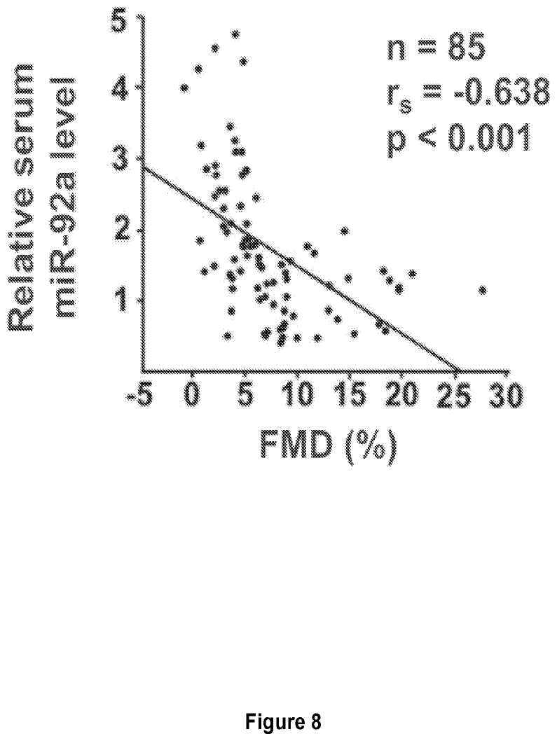

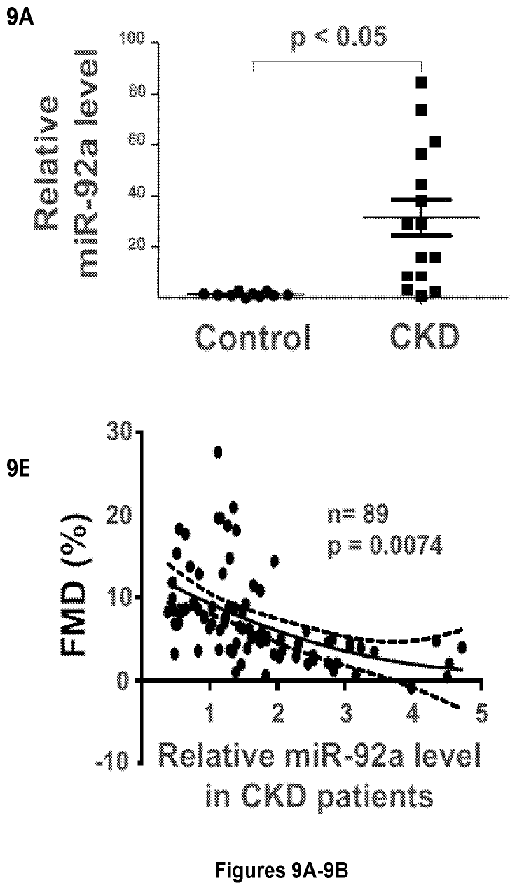

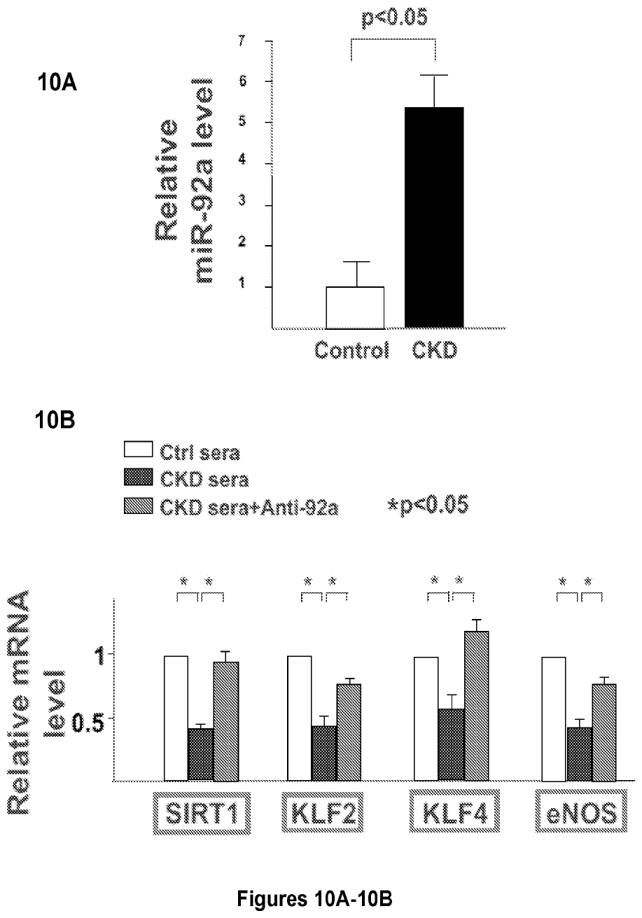

Studies have shown that the CKD milieu increases serum miR-92a levels in patients and that serum miR-92a was likely derived from endothelial cells (ECs) (Shang et al. JASN, 2017). ECs cultured with CKD serum increased their miR-92a expression and had reduced levels of several molecules critical for maintaining endothelial homeostasis, including sirtuin-1, Kruppel-like factors 2 and 4, and endothelial nitric oxide synthase (FIG. 10), which can lead to a decreased flow mediated dilation (FIG. 6). Inhibiting miR-92a rescued those vasoprotective molecules in cultured ECs in the CKD milieu (FIG. 10).

To date, there are three clinically relevant delivery platforms for modulating gene and miRNA expression using therapeutic nucleotides: (i) naked therapeutic nucleotides, (ii) lipid nanoparticles, and (iii) conjugate nucleotide systems. Yet they suffer from moderate to serious disadvantages, including rapid degradation, immunogenicity, poor circulation half-life, high cytotoxicity, and poor cellular uptake.

The significance of the present disclosure includes at least two aspects. First, it provides novel nanomedicine approaches to treat vascular disorders with unmet medical need. Second, it integrates targeted nanomedicine and RNA therapeutics to create a new avenue for the treatment of various vascular diseases including stabilization of AFV or vein grafts, restenosis, and atherosclerosis. This disclosure further provides, in part, a peptide-targeted polyelectrolyte complex using micelles to deliver therapeutic nucleotides to vascular cells, promoting AVF maturation and reducing AVF failure, reducing restenosis, and reducing atherosclerotic lesions.

Compositions

In some embodiments, pharmaceutical compositions contemplated herein include a therapeutically effective amount of a targeted nanoparticle including one or more inhibitors of endothelial inflammation, such as, for example, an miR-92a inhibitor. Such compositions may further include an appropriate pharmaceutically acceptable carrier, solvent, adjuvant, diluent, or any combination thereof. The exact nature of the carrier, solvent, adjuvant, or diluent will depend upon the desired use (e.g., route of administration) for the composition, and may range from being suitable or acceptable for veterinary uses to being suitable or acceptable for human use.

In some embodiments, pharmaceutical compositions contemplated herein include one or more nanoparticles that carry the one or more miR-92a inhibitors, for example, inside the nanoparticle, attached to an external surface of the nanoparticle, or both. In some embodiments, the nanoparticles include one or more targeting moeities attached thereto to enable targeted delivery of the nanoparticle to a desired location. For example, the targeting moeity can target the nanoparticle to a site of endothelial inflammation associated with a vascular disease or disorder or wound.

Any miR-92a inhibitor is contemplated herein. For example, contemplated miR-92a inhibitors include those available from Dharmacon. Other contemplated miR-92a inhibitors include custom miRIDIAN Hairpin Inhibitor (hsamiR-92a-3p, MIMAT0000092; Ref #IH-300510-06).

Such compositions optionally include secondary therapeutic agents (possibly also carried on or in contemplated nanoparticles).

miR-92a inhibitors of the present disclosure can be administered through a variety of routes and in various compositions. For example, pharmaceutical compositions containing miR-92a inhibitors can be formulated for oral, intravenous, topical, ocular, buccal, systemic, nasal, injection, transdermal, rectal, or vaginal administration, or formulated in a form suitable for administration by inhalation or insufflation. In some embodiments of the present disclosure, administration is oral or intravenous.

A variety of dosage schedules is contemplated by the present disclosure. For example, a subject can be dosed monthly, every other week, weekly, daily, or multiple times per day. Dosage amounts and dosing frequency can vary based on the dosage form and/or route of administration, and the age, weight, sex, and/or severity of the subject's disease. In some embodiments of the present disclosure, one or more miR-92a inhibitors is administered orally, and the subject is dosed on a daily basis.

The therapeutic agents (also referred to as “compounds” herein) described herein (e.g., miR-92a inhibitors and secondary therapeutic agents), or compositions thereof, will generally be used in an amount effective to achieve the intended result, for example, in an amount effective to provide a therapeutic benefit to subject having the particular disease being treated. As used herein, therapeutic benefit refers to the eradication or amelioration of the underlying disease being treated and/or eradication or amelioration of one or more of the symptoms associated with the underlying disease such that a subject being treated with the therapeutic agent reports an improvement in feeling or condition, notwithstanding that the subject may still be afflicted with the underlying disease.

Non-limiting examples of contemplated secondary therapeutic agents include those that can promote vascular health, inhibit vascular inflammation, promote endothelial health, suppress smooth muscle proliferation/restenosis, reduce thrombosis, reduce oxidate stress, suppress smooth muscle proliferation/restenosis, reduce excessive, abnormal, or imbalanced degradation and synthesis of extracellular matrix, and promote the health and functional phenotype of endothelial cells and vascular smooth muscles. Examples of agents to promote vascular health can include anticoagulants (blood thinners), antiplatelet agents, angiotensin-converting enzyme (ACE) inhibitors, angiotensin II receptor blockers, angiotensin-receptor neprilysin inhibitors, beta blockers, calcium channel blockers, cholesterol-lowering medications, digitalis preparations, diuretics, and vasodilators. Further therapeutic agents contemplated for use herein include anti-inflammatory medications. Still further therapeutic agents contemplated for use herein include IL-1b blockers and inflammasome blockers. Other examples of inhibitors of vascular inflammation include dehydroepiandrosterone sulfate, inhibitors of myeloperoxidase, inhibitors of dipeptidyl peptidase-4 (DPP-4), inhibitors of inflammasome, activators of nitric oxide synthase, and inhibitor of small GTPAse RhoA.

Determination of an effective dosage of compound(s) for a particular disease and/or mode of administration is well known. Effective dosages can be estimated initially from in vitro activity and metabolism assays. For example, an initial dosage of compound for use in a subject can be formulated to achieve a circulating blood or serum concentration of the metabolite active compound that is at or above an IC₅₀ of the particular compound as measured in an in vitro assay. Calculating dosages to achieve such circulating blood or serum concentrations taking into account the bioavailability of the particular compound via a given route of administration is well within the capabilities of a skilled artisan. Initial dosages of compound can also be estimated from in vivo data, such as from an appropriate animal model.

Dosage amounts of miR-92a inhibitors and secondary therapeutic agents can be in the range of from about 0.0001 mg/kg/day, about 0.001 mg/kg/day, or about 0.01 mg/kg/day to about 100 mg/kg/day, but may be higher or lower, depending upon, among other factors, the activity of the active compound, the bioavailability of the compound, its metabolism kinetics and other pharmacokinetic properties, the mode of administration and various other factors, including particular condition being treated, the severity of existing or anticipated physiological dysfunction, the genetic profile, age, health, sex, diet, and/or weight of the subject. Dosage amounts and dosing intervals can be adjusted individually to maintain a desired therapeutic effect over time. For example, the compounds may be administered once, or once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of compound(s) and/or active metabolite compound(s) may not be related to plasma concentration. Skilled artisans will be able to optimize effective dosages without undue experimentation.

For example, a dosage contemplated herein can include a single volume of about 0.1, 0.2, 0.3, 0.4, 0.5, 0.6, 0.7, 0.8, 0.9, 1.0, 1.5, 2.0, 2.5, or 3.0 mL of a pharmaceutical composition having a concentration of a miR-92a inhibitor at about 0.00001, 0001, 0.001, 0.01, 0.05, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 10, 15, 20, 50, 100, 200, 500, or 100 mM in a pharmaceutically acceptable carrier.

Nanoparticles

The present disclosure contemplates use of polyelectrolyte micelles (also referred to as nanoparticles herein) to deliver therapeutic agents. Polymers that bear charge in an aqueous environment are called polyelectrolytes. When oppositely charged polymers are mixed under the right conditions, they form complexes. Polyelectrolytes, including at least one attached to a non-charged, water soluble block, can be mixed at a stoichiometric charge ratio with an oppositely charged homopolymer to form particles of a relatively compact core surrounded by a dilute corona of neutral water soluble block. These nanometer-sized particles are called polyelectrolyte complex micelles, polyion complex micelles, interpolyelectrolyte complex micelles, complex coacervate core micelles, or polyelectrolyte micelles. Polyelectrolyte complexes composed of nucleic acids and positively charged polymers have been explored as a possibility to neutralize the charge on the molecule and protect it from enzymatic degradation. Polyelectrolyte complex micelles have great potential as gene delivery vehicles because of their ability to encapsulate charged nucleic acids, forming a core by neutralizing their charge, while simultaneously protecting the nucleic acids from non-specific interactions and enzymatic degradation. Furthermore, to enhance specificity and transfection efficiency, polyelectrolyte complex micelles can be modified to include targeting capabilities.

The contemplated polyelectrolyte micelles can comprise polyethylene glycol (PEG) domains as well as domains of positively charged amino acids (e.g., repeated positively charged amino acids). PEG domains prevent macrophase separation, stabilizing the micelles. The domains further protect the nanoparticles from recognition by the reticuloendothelial system in the body. The PEG domain can be comprised of PEG having an average molecular weight of about 1,000 to about 100,000 Daltons (Da).

The micelles can comprise a domain of positively charged amino acids, such as lysine, arginine, and/or histidine. This domain can complex with negatively charged therapeutic agents, such as miRNA inhibitors. The domain of repeated positively charged amino acids can include about 2 to about 100 residues.

Contemplated nanoparticles for use herein include, for example, polyelectrolyte complex micelles that can effectively incorporate negatively-charged nucleotides in the core and functionally display tissue-targeting peptides on the surface. These self-assembled nano-scale carriers (˜20 nm in diameter) are formed by electrostatic interaction between two oppositely-charged polymers. Polyethylene glycol (PEG)-2000 was conjugated with poly-lysine on one side to form positively-charged building blocks and conjugated with targeting peptides on the other side to functionalize the nanoparticles to bind specific cell membrane molecules. Negatively-charged nucleotides are neutralized by poly-lysine and encapsulated in the cores of the nanoparticles. This approach offers multiple advantages, including: (i) the nano-scale of micelles significantly increases the surface area:volume ratio that can enhance specific targeting, and (ii) the self-assembling feature of the polyelectrolyte micelles eliminates the use of chemical cross-linking agents, thereby reducing possible toxicities.

Additional micelles are contemplated for use herein, such as those disclosed in International Application No. PCT/US2006/020760, Vieregg et al. (J. Am. Chem. Soc. 2018, 140, 1632-1638), Lueckheide et al. (Nano Lett. 2018, 18, 7111-7117), and Marras et al. (Polymers 2019, 11, 83), each of which is incorporated by reference.

Targeting Molecules

The present disclosure contemplates use of targeting molecules (or targeting moieties) with the nanoparticles disclosed herein for targeted delivery of therapeutic compositions, such as miR-92a inhibitors, or for incorporation into pharmaceutical compositions as described herein. Targeting molecules can include peptides such as CGVHPKQHR (SEQ ID NO: 6) or VHPKQHR (SEQ ID NO: 2), which were identified via phage display and allows for targeting of vascular endothelial cells through VCAM-1. Peptide targeting molecules further include the amino acid sequence NNQKIVNLKEKVAQLEA (SEQ ID NO: 3), which allows for the targeting of intercellular adhesion molecule 1 (ICAM-1). ICAM-1 is a cell surface glycoprotein typically expressed on endothelial cells. Peptide targeting molecules further include the amino acid sequence DITWDQLWDLMK (SEQ ID NO: 4), which allows for the targeting E-selectin. E-selectin is a cell adhesion molecule expressed only on cytokine-activated endothelial cells. In another embodiment, the targeting molecule is a peptide comprising the amino acid sequence REKA (SEQ ID NO: 1) or the peptide comprising the amino acid sequence CREKA (SEQ ID NO: 5), both of which bind fibrin. CGSPGWVRCG (SEQ ID NO: 7) is a peptide identified by phage display to bind specifically to lung endothelial cells and can be displayed on this nanoparticle.

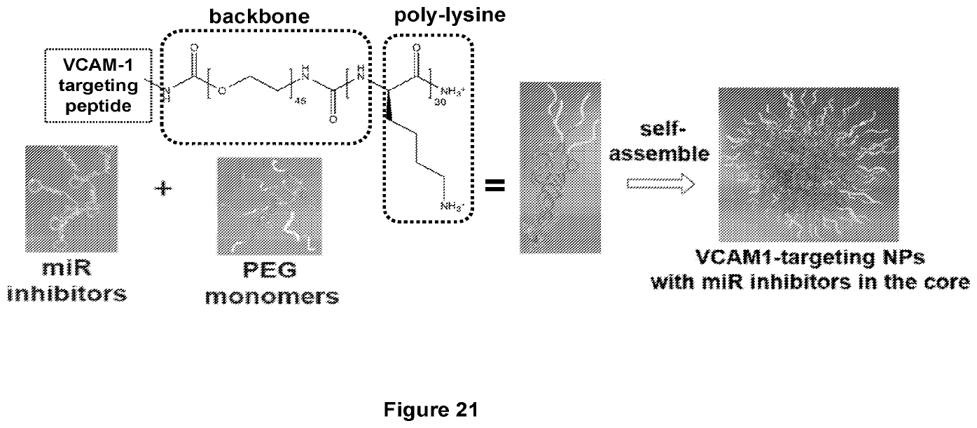

The expression of vascular cell adhesion molecule 1 (VCAM1) is low in healthy endothelium but increases in inflamed endothelial cells (ECs). To achieve effective targeting to VCAM1-expressing ECs, the micelles are functionalized with a VCAM1 binding peptide that has been shown to facilitate VCAM1-mediated intracellular internalization of nano-materials in endothelium in vitro and in vivo (FIG. 21). In some embodiments, contemplated targeting peptides are positioned at the periphery of the corona of the nanoparticles.

In one embodiment, a contemplated targeted nanoparticle containing an miR-92a inhibitor (2 μM) is VHPKQHR-PEG-K30. In one embodiment, a contemplated targeted nanoparticle containing an miR-92a inhibitor (2 μM) is CGVHPKQHR-PEG-K30. In one embodiment, a contemplated targeted nanoparticle containing an miR-92a inhibitor (2 μM) is NNQKIVNLKEKVAQLEA-PEG-K30. In one embodiment, a contemplated targeted nanoparticle containing an miR-92a inhibitor (2 μM) is DITWDQLWDLMK-PEG-K30. In one embodiment, a contemplated targeted nanoparticle containing an miR-92a inhibitor (2 μM) is REKA-PEG-K30. In one embodiment, a contemplated targeted nanoparticle containing an miR-92a inhibitor (2 μM) is CREKA-PEG-K30. In one embodiment, a contemplated targeted nanoparticle containing an miR-92a inhibitor (2 μM) is CGSPGWVRCG-PEG-K30.

In some embodiments, contemplated nanoparticles containing an miR-92a inhibitor exhibit a polydispersity of about 0.1 to about 0.3.

In some embodiments, contemplated nanoparticles containing an miR-92a inhibitor contained with the core exhibit a spherical shape and have a diameter (in nanometers, nm) of about 10, 11, 12, 13, 14, 15, 16, 17, 18, 19, 20, 21, 22, 23, 24, 25, 30, 35, 40, 45, or 50 nm.

The nanoparticle delivery system of the present disclosure possesses multiple advantages compared with other nanoparticle-based platforms. The first advantage is higher stability: the therapeutically active components (e.g., miR-92a inhibitors or other nucleic acid based therapeutics) can be encapsulated in the inner core of the polyelectrolyte complex micelles and can therefore be protected by the outer layer of biocompatible polymers. Through this approach, first, the degradation of nucleotides of an miR-92a inhibitor by serum nucleases is prevented; second, the micelles are capable of escaping renal clearance; and third, the immunogenic responses are avoided. The second advantage is higher safety: cell-targeting peptides (e.g., those targeting to Vascular Cell Adhesion Molecule 1 (VCAM-1)) are covalently conjugated on the periphery of the polyelectrolyte complex micelles, which significantly reduces cytotoxicity and increases circulation time by circumventing nonspecific interaction with serum components. The third advantage is higher specificity: with the defined chemical structures of the targeting peptides, the polyelectrolyte complex micelles are able to bind specific receptors and penetrate targeted cells. The final advantage is higher scalability. This approach does not require chemical modifications on nucleotides for conjugation, nor does it need to engineer hard-to-reproduce lipid nanoparticles. The synthesis of the core components in the micelles is highly automated. In addition, the targeting peptides are easily changeable to target different receptors.

Further, as shown herein, the use of targeted nanoparticles permits use of a lower amount of a therapeutic agent for the treatment of a vascular disorder or wound due to the specific targeting of the therapeutic agent to the site of the vascular disorder or wound. In this way, use of targeted nanoparticles can significantly lower the dosage of a therapeutic agent required to treat a vascular disorder or wound, which can significantly reduce costs associated with the treatment. For example, a therapeutically effective amount of a therapeutic agent to be delivered by a targeted nanoparticle can be at least about 10, 20, 30, 40, or 50% lower than the therapeutically effective amount of the naked (non-targeted) therapeutic agent.

Methods

In some embodiments, methods of treating and/or preventing a vascular disorder in a subject in need thereof include administering to the subject a therapeutically effective amount of one or more miR-92a inhibitors and optionally a secondary therapeutic agent. Treatable and/or preventable vascular disorders can include arteriovenous fistula (AVF) failure, stenosis, restenosis, and atherosclerosis.

In some embodiments, therapeutic methods contemplated herein can also treat and/or prevent complications associated with or promote endothelial wound healing (e.g., caused by trauma or surgery) by administering to the subject a therapeutically effective amount of one or more miR-92a inhibitors and optionally a second therapy and/or secondary therapeutic agent. For example, treatment and/or prevention of complications associated with endothelial wound healing is associated with the reduction of inflammation at the site of the wound and the stimulation of endothelial growth.

In some embodiments, therapeutic methods contemplated herein can also accelerate endothelial growth to treat wound healing (e.g., caused by trauma or surgery) by administering to the subject a therapeutically effective amount of one or more miR-92a inhibitors and optionally a second therapy and/or secondary therapeutic agent.

AVF failure addressed by the present disclosure can be of multiple types. These types can include maturation failure, in which a newly created AVF does not mature sufficiently (such as does not have adequate open lumen size or blood flow rate) to be used for dialysis. The failure can also be one of durability, which means that an AVF is able to be used for dialysis, but then later on develops problems (such as stenosis) and cannot be used. These failures can result from pathological vascular remodeling, insult to the endothelial layer, stenosis, and thrombosis.

The AVFs contemplated by the present disclosure can be of a variety of subtypes. The subtypes can include forearm AVF (e.g., snuff-box, distal radiocephalic or transposed radiobasilic), proximal forearm AVF (e.g., proximal radiocephalic, perforator-combinations), brachiocephalic AVF, the brachial artery-to-transposed basilic vein fistula, and lower extremity AVF.

The present disclosure contemplates a variety of methods of administering the therapeutic agents, targeting molecules, and micelles disclosed herein, including local, oral, nasal, rectal, intravaginal, topical, subcutaneous, intradermal, intramuscular (IM), intravenous (IV), intrathecal (IT), intracerebral, epidural, or intracranial administration. Local, in situ administration of these compositions is contemplated.

The present disclosure contemplates methods that result in a variety of indications of improvement for the AVF. These indications can include a greater lumen cross-sectional area, a greater lumen diameter, and/or increased flow rate.

The present disclosure contemplates use of the disclosed methods in conjunction with other treatments for failure of AVF. These other treatments can include percutaneous transluminal angioplasty or endovascular declotting techniques. Other inventions are contemplated, such as out-patient interventional procedure with angioplasty and/or stent placement.

### EXAMPLES

The Examples that follow are illustrative of specific embodiments of the disclosure, and various uses thereof. They are set forth for explanatory purposes only and should not be construed as limiting the scope of the disclosure in any way.

### Example 1: Formation and Characterization of Micelles

Introduction

Engineered polyelectrolyte complex micelles were produced to inhibit dysregulated vascular miRNAs that promote AVF. The approach is to encapsulate miRNA inhibitors in the core of the micelle and use peptides on the periphery of the nanoparticle corona to target markers. One strategy targets vascular cell adhesion molecule 1 (VCAM-1) on the surface of endothelial cells using micelles conjugated with VCAM-1-binding peptides that have the sequence valine-histidine-proline-lysine-glycine-histidine-arginine, or VHPKQHR (SEQ ID NO: 2). The formation of these miRNA inhibitor-containing, peptide-targeted micelles is achieved via the electrostatic complexation between the negatively charged miRNA inhibitors and polycations containing targeting peptides that has been designed and synthesized (FIG. 21). Both polycation molecules contain three functional domains consisting of targeting peptides for cellular or plaque localization, a polyethylene glycol (PEG) domain to prevent macrophase separation, and a polylysine domain to complex with the negatively charged miRNA inhibitors. Combining the miRNA inhibitors with these targeting peptide-PEG2000-polylysine molecules should result in the formation of electrostatically driven, self-assembled micelles with a polylysine-miRNA inhibitor core, protected by a PEG corona that is decorated with the targeting peptide. By taking advantage of the benefits of self-assembly, the micelle corona can be tailored to enable the targeting of diverse cell types and load the micelles with specific miRNA inhibitors, thereby providing the means to target various pathological mechanisms in a wide range of cells and contexts.

Methods

Material Synthesis and Purification

Targeting peptide-PEG(2000)-poly-L-lysine with a degree of polymerization of 30 (Peptide-PEG-K30) was synthesized using standard fluorenylmethyloxycarbonyl(FMOC) solid phase synthesis methods on an automated PS3 peptide synthesizer from Protein Technologies, Inc. (Tucson, AZ, USA). FMOC protected amino acids were also purchased from Protein Technologies Inc. The heterobifunctional molecule, FMOC-PEG2000-COOH, was purchased from JenKem Technology USA (Allen, TX, USA). A peptide targeting VCAM-1 which has the sequence VHPKQHR (SEQ ID NO: 2) was used in this study. The targeting peptide-PEG-K30 molecule was subsequently purified using reverse phase high performance liquid chromatography (HPLC, Shimadzu Corporation, Japan) and confirmed using a Bruker UltrafleXtreme (Fremont, CA, USA) matrix-assisted laser desorption/ionization time of flight mass spectrometry (MALDI-TOF). The miRNA inhibitor molecules (miRIDIAN microRNA Hairpin Inhibitors, Dharmacon, USA) are single-stranded, chemically enhanced oligonucleotides diluted to a working stock solution of 100 μM.

Micelle Formation

Micelles of varying concentrations were formed by complexing the targeting-peptide-PEG-K30 and miRNA inhibitors at an equal charge molar ratio. First, the appropriate amount of deionized water was added, followed by the miRNA inhibitor, and finally the targeting-peptide-PEG-K30. The sample was vortexed after each polymer addition. The complexing of targeting-peptide-PEG-K30 with miR inhibitors was performed under charge neutral conditions in which the number of charged monomer units from the polylysine equaled the number of charged units in the miR inhibitors. Micelles were made by complexing VHPKQHR-PEG-K30 with miR-92a inhibitors and miR inhibitor controls. DLS was used to confirm the formation and measure the hydrodynamic diameter of all micelles. The size of polyelectrolyte complex micelles is determined by the length of the charged block covalently linked to the neutral non-charged polymer, as well as the ratio between them.

Results: Characterization of Micelles

Dynamic Light Scattering (DLS)

Micelle solutions containing 5 μM miRNA inhibitors were measured at 90° using a BI-200SM goniometer containing a red laser diode with a wavelength of 637 nm and TurboCorr digital correlator, all from Brookhaven Instruments (Holtsville, NY, USA). Brookhaven Instruments Dynamic Light Scattering software was used to analyze the inverse Laplace transforms of the intensity autocorrelation functions using the non-negatively constrained least-squares (NNLS) algorithm to obtain multimodal size distribution data.

Transmission Electron Microscopy (TEM)

Negatively stained TEM samples were prepared by placing micelles on 400 mesh lacey carbon grids (Ted Pella, Redding, CA, USA) and then staining with a 1 wt % uranyl acetate solution. Images were obtained using a FEI Tecnai F30 electron microscope operated at 300 kV.

Zeta Potential

Zeta potential was determined by measuring the polyanions: miR92a inhibitor, miR33a inhibitor, and miR inhibitor control at a concentration 4 μM and the polycations VHPKQHR-PEG-K30 at a concentration of 9 μM (equal in molar charge to the miRNA inhibitors). Micellar solutions of the combined polyanion and polycation were measured at the combined concentration of 13 μM (equal molar charge). All samples were dissolved in MQ water and measured at 25° C. (Zetasizer Nano ZS, Malvern, Worcestershire, United Kingdom, N=3).

Treatment Before Administration

To separate unbound, free polymers from the micelle preparation, the micelle solution was separated into filtered and concentrated fractions by a 50-kDa cut-off membrane (Amicon® Ultra-0.5 centrifugal filter devices, Millipore). The concentrated fraction was further washed with nuclease-free water and the volume of concentrate was evaluated after centrifugation and concentration of concentrated fraction calculated.

Only slight variations in the size of all the micelle constructs were expected, and the results shown in Table 1 confirms this result.

TABLE 1
Dynamic light scattering of compositions.

| Micelle | Dₕ(nm) | Polydispersity |
|---|---|---|
| VHPKQHR-micelle miR-92a inhibitor | 18.2 | 0.103 |
| VHPKQHR-micelle miR inhibitor control | 15.5 | 0.257 |

Dₕ-particle diameter.

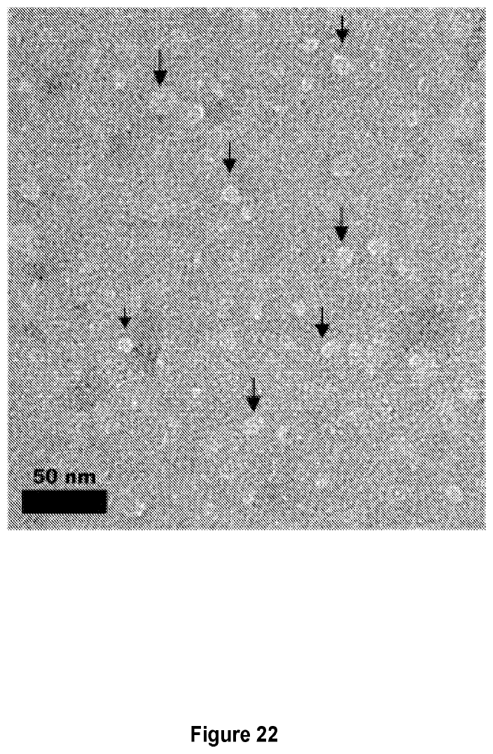

The average size of all the micelles was 21.1±4 nm. Negatively stained TEM confirmed the DLS observations. A characteristic TEM image is shown in FIG. 22. Micelles between 15-20 nm in diameter were observed using TEM, which is in agreement with the DLS results. As another characterization method, the zeta-potential of the individual polymers prior to complexation and the micelle solutions was measured. The results, shown in Table 2, indicate that the targeting-peptide-PEG-K30 molecules are highly positively charged and the miR inhibitors are highly negatively charged.

TABLE 2
Zeta potential of compositions.

| | Zeta |
|---|---|
| Polymer or Micelle | potential |
| miR-92a inhibitor | −29.6 ± 1.9 |
| miR-33a inhibitor | −22.2 ± 2.9 |
| VHPKQHR-PEG-K30 | 30.9 ± 3.1 |
| VHPKQHR-micelle miR-92a inhibitor | 7.2 ± 1.0 |
| VHPKQHR-micelle miR inhibitor control | 18.3 ± 2.1 |

These results were expected since targeting-peptide-PEG-K30 contains positively charged poly-L-lysine and miR inhibitors contain the negatively charged phosphate backbone. The micellar solutions all have values in between the miR inhibitors and the peptide-PEG-K30 molecules indicative of a mixture of the two components.

### Example 2: Inhibition of microRNA-92a and Effects on Pathological Vascular Remodeling Under Disturbed Flow—AVF Maturation Failure

Introduction

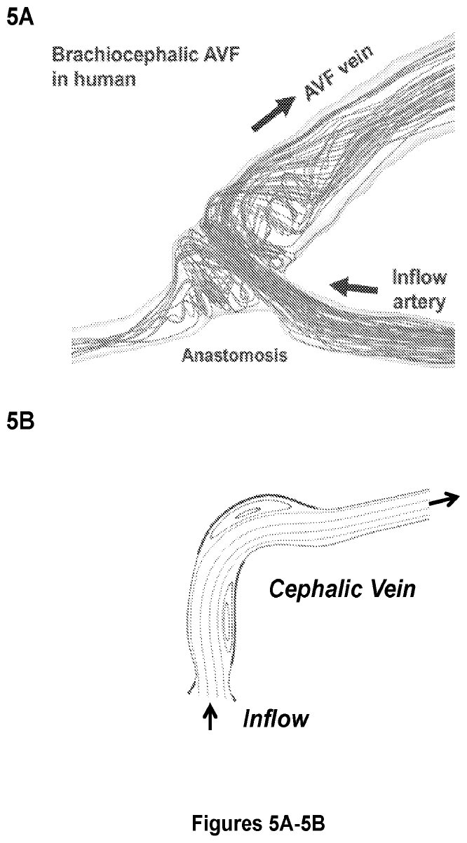

The most common causes of AVF failure are activation of endothelium, neointimal hyperplasia, stenosis, and thrombosis at the arteriovenous anastomosis and cephalic arch, both sites are exposed to complex hemodynamics after AVF creation. (See FIG. 2). Blood flow in veins is typically steady, slow, and non-pulsatile under normal physiological conditions but local vein geometry and flow parameters are drastically remodeled after access creation. At the sites of arteriovenous anastomosis and cephalic arch, increased flow rate and enhanced pulsatility due to AVF creation impose complicated patterns of multidirectional hemodynamics at variable frequencies leading to fluid disturbance featuring oscillation, flow reversal, or recirculation. This complex hemodynamics, or disturbed flow, has been linked to the endothelial activation and consequent pathological vessel remodeling leading to a wide range of vascular diseases, comprising atherosclerosis and AVF failure. FIG. 5A depicts the complex hemodynamics at the arteriovenous anastomosis (juxta-anastomotic venous segment in humans) after brachiocephalic AVF creation in a hemodialysis patient (FIG. 5B). Here, the in vivo potency of the proposed targeted nanomedicine approach in alleviating neointimal hyperplasia was determined in the carotid artery-jugular vein AVF mouse model.

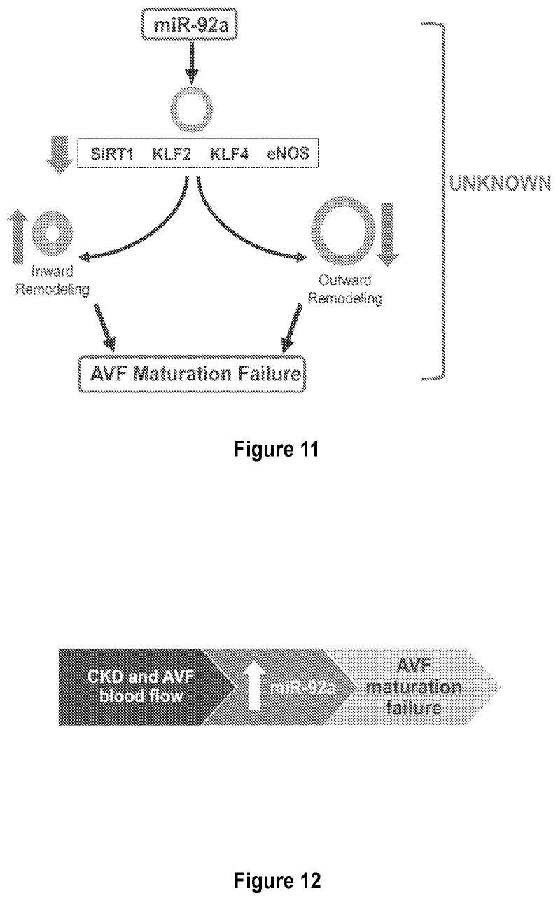

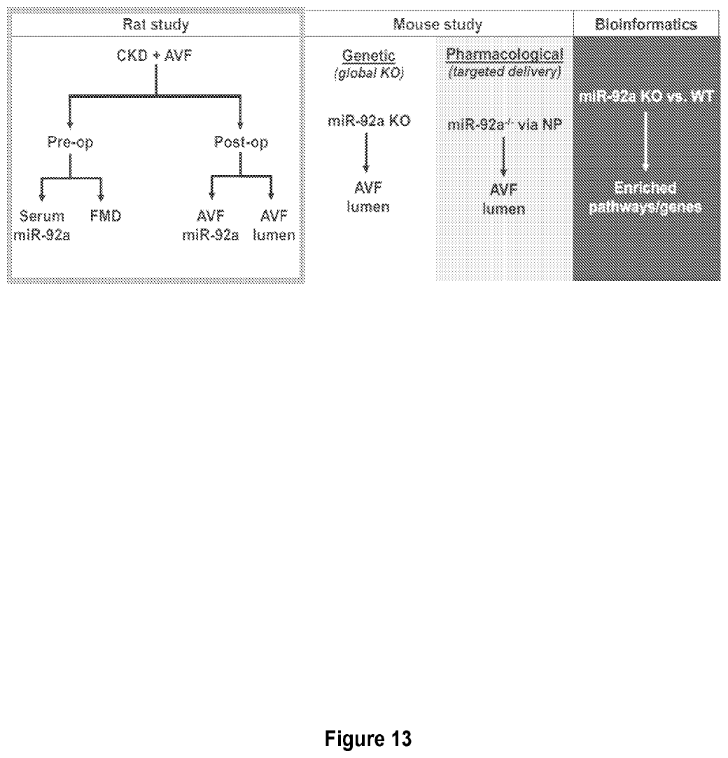

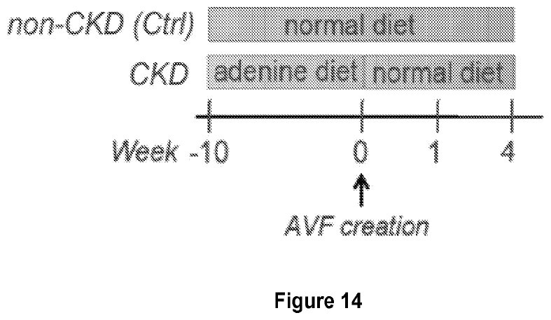

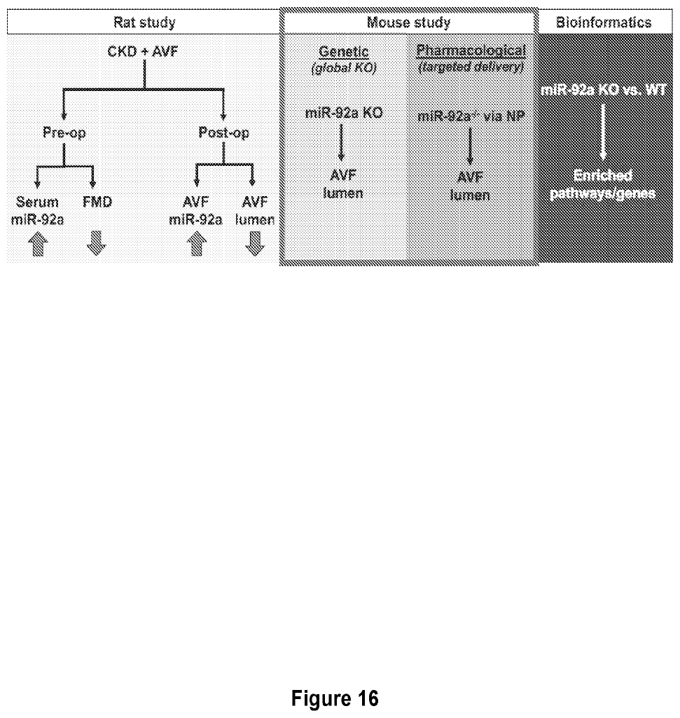

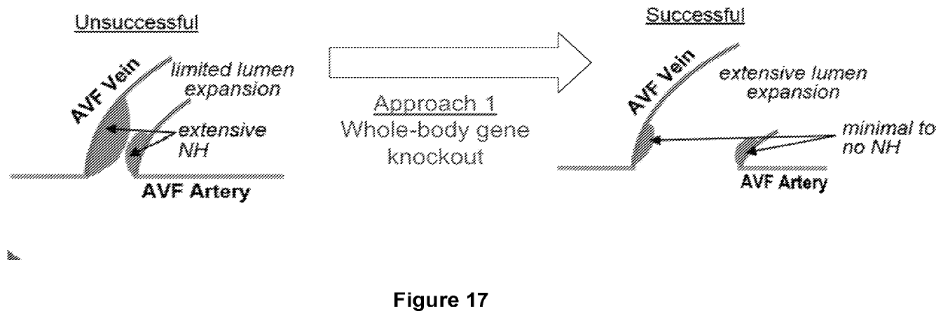

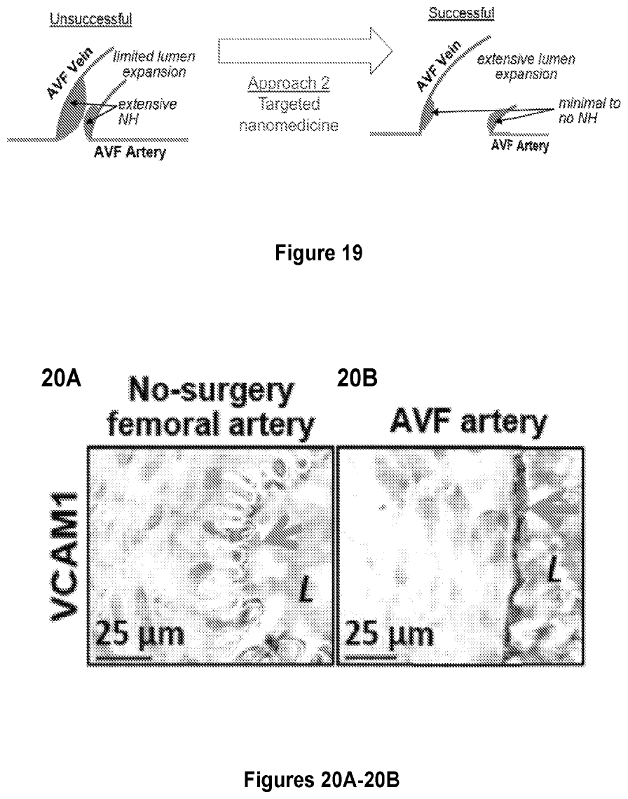

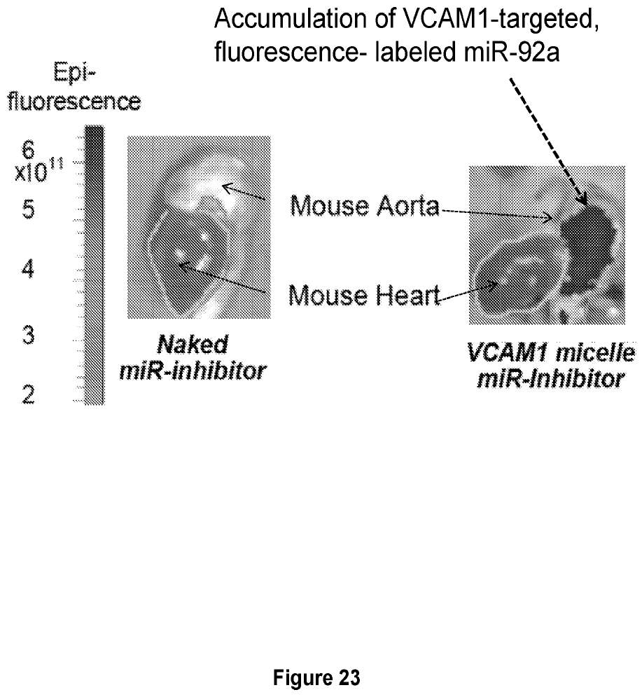

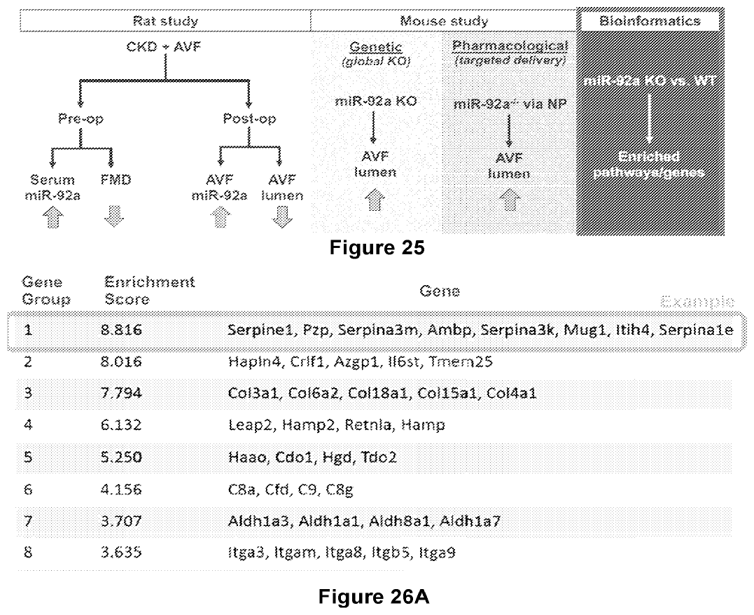

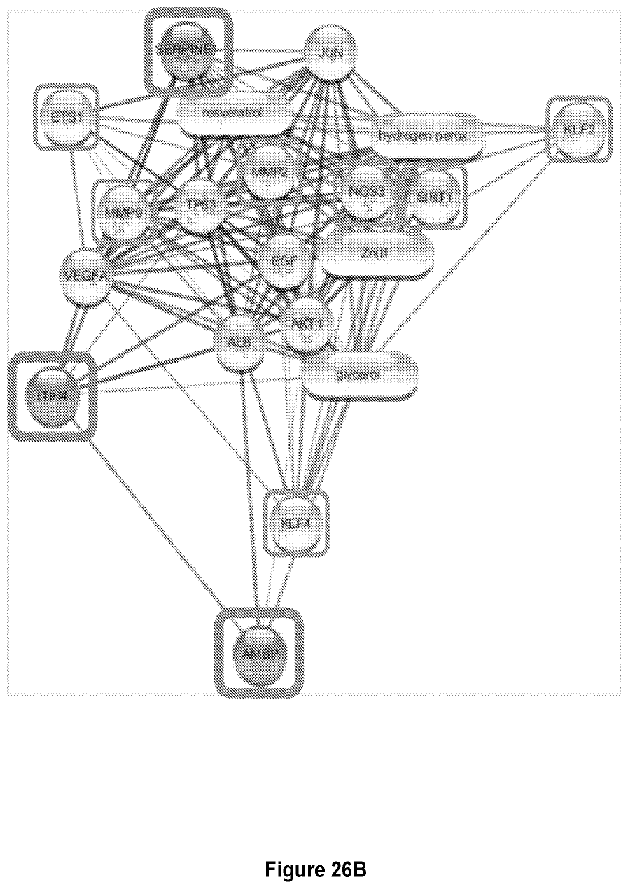

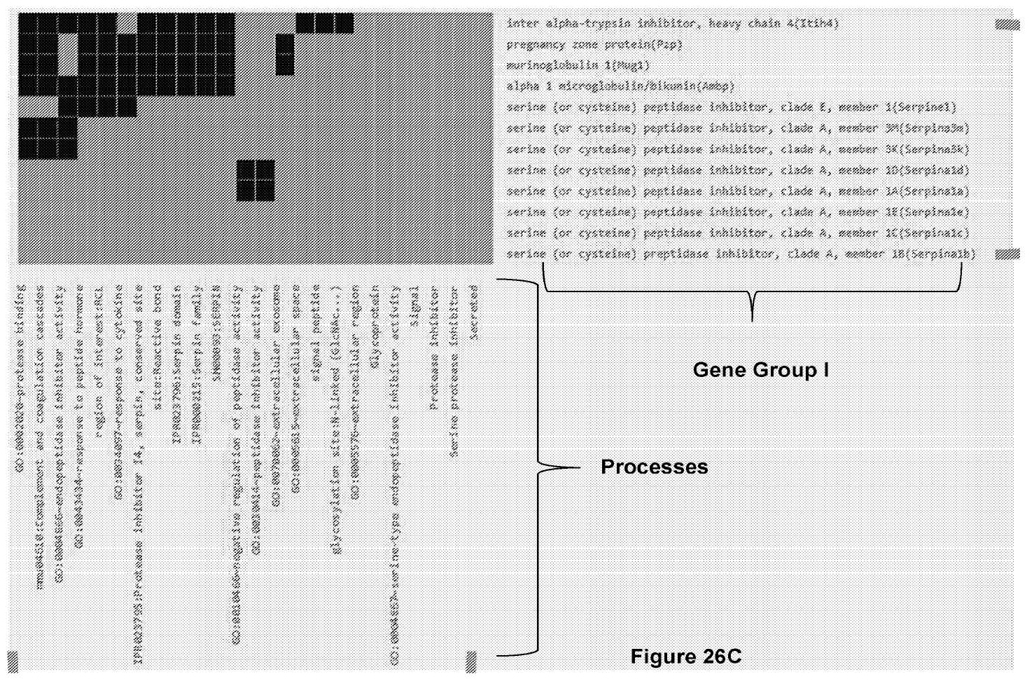

Further, a previous study showed that endothelial health was associated with the development of arteriovenous fistulas (AVFs) (Allon et al. JASN, 2016). MicroRNA (miR)-92a is a major contributor to vascular endothelial dysfunction. It has been previously reported that patients with CKD have increased serum miR-92a levels when compared to non-CKD control subjects, and that serum miR-92a is likely derived from the endothelium (Shang et al. JASN, 2017). Thus, the relationship between miR-92a and AVF development in animal models was investigated (FIGS. 11-26).

Methods

In young male Wistar rats with normal kidney function or with adenine diet-induced CKD, femoral AVFs were created and then AVF lumen diameter was assessed by ultrasound and AVF tissue miR-92a levels by RT-PCR at 4 weeks after creation, as described in Langer et al. Kindey International 2010. In a mouse carotid-jugular AVF model as described in Chun et al. J Vis Exp 2016, miR-92a inhibition was achieved using genetic and pharmacological approaches: (1) whole-body knockout (miR-92a−/−) with C57BL/6 mice used as wild-type (WT) controls; or (2) nanoparticles (NPs) that encapsulate miR-92a inhibitors and target inflamed endothelium as described in Example 1 (unencapsulated (naked) miR-92a inhibitors and saline were used as no-NP and no-treatment controls, respectively).

The anastomosis angles of mouse carotid-jugular AVFs (approximately 70-90°) are similar to human brachiocephalic AVFs in the literature, and this mouse AVF model is well established for the study of human AVF maturation failure and drug targets. Ten-week old male C57BL6 mice received the inhibitor treatment (8 mg/kg body weight) intravenously, through the tail vein, at 1 day after AVF creation and were sacrificed 1 week later. Mouse AVF cross-sectional lumen area and the area of neointimal hyperplasia were quantified by histology using Image J.

Results

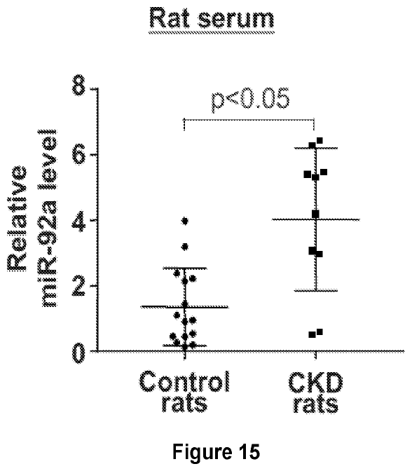

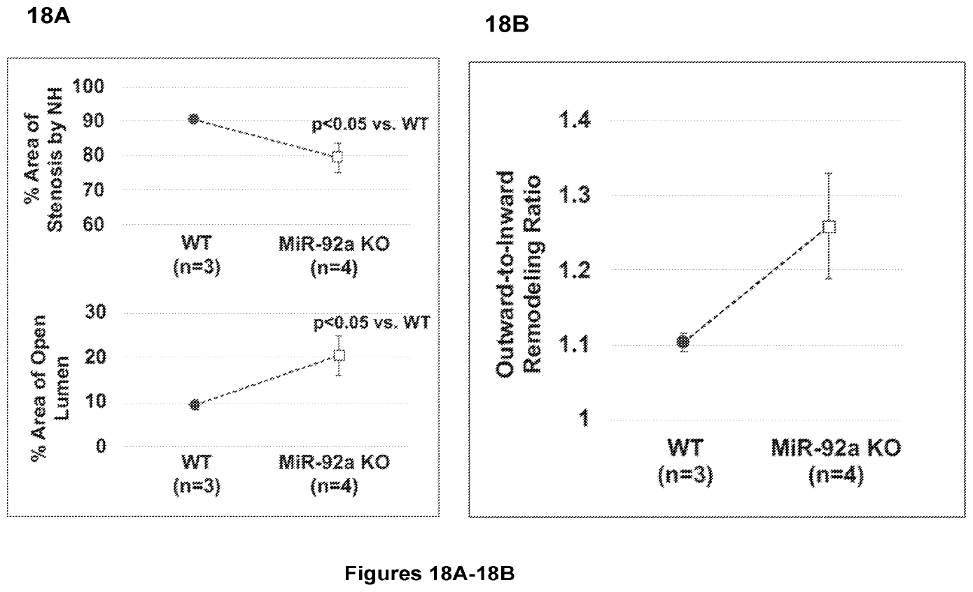

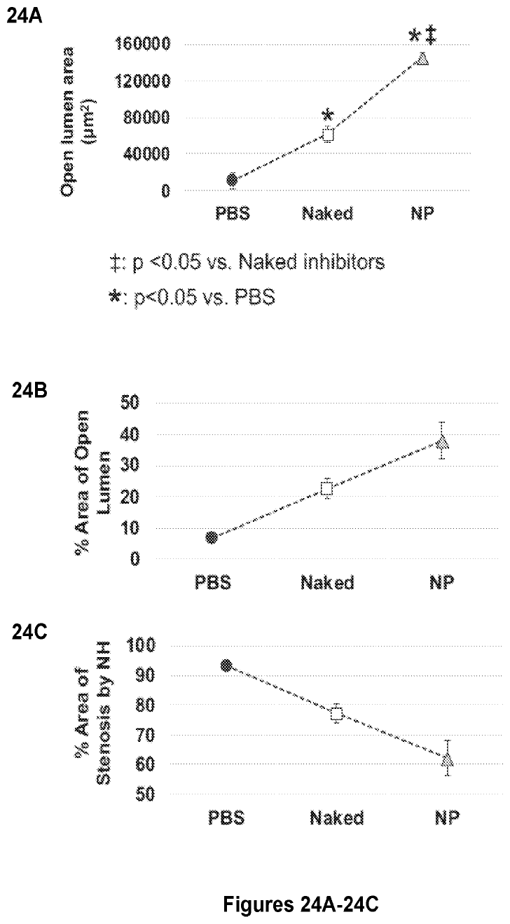

When compared to AVFs in non-CKD rats, AVFs in CKD rats had increased miR-92a in serum (3 fold, p<0.05) (FIG. 15). In the knockout study, the percent open lumen area of AVF veins was larger in miR-92a−/− mice (72% of total area) than in WT mice (12%) (FIGS. 18A and 18B). In the inhibitor study, both NP-encapsulated (41%) and un-encapsulated (23%) miR-92a inhibitors resulted in larger open lumen area than saline control (5%), and the effect of encapsulated inhibitors was more pronounced (FIG. 24B). It was determined that while both encapsulated and naked miR-92a inhibitors resulted in greater open lumen area and smaller NH area when compared to the PBS control, the effect of encapsulated inhibitors was more pronounced (FIG. 24A).

In the mouse model, inhibition of miR-92a improved AVF development (FIG. 24). Thus, this nanomedicine approach may offer a novel and effective therapy to enhance AVF maturation in CKD patients.

Conclusion

These experiments confirm that VCAM-1-targeted polyelectrolyte complex micelles effectively deliver miR-92a inhibitors preferentially to inflamed endothelial cells. The experiments sought to determine the therapeutic effectiveness of VCAM-1-targeted polyelectrolyte complex micelles in inhibiting endothelial miR-92a and suppressing pathological AVF vascular remodeling in vivo. These studies should further preclinical development, and perhaps clinical testing, of a new therapeutic strategy to treat AVF failure, a still critically important disease with unmet medical need.

Results from the experiments disclosed herein indicate that combination of miRNA therapies and targeted nanomedicine is an attractive new strategy to provide preventative maintenance to AVF placements. First, prior data employing cultured cells, animal models (mouse and swine), and human genetics identified that increased expression of endothelial miR-92a is a major molecular signature of endothelial activation and pathological vascular remodeling under disturbed flow. Second, genetic deletion of miR-92a in experiments described herein significantly lessened AVF failure in a mouse model. Third, the novel nanoparticles described here preferentially target inflamed endothelial cells and moreover, intracellularly deliver miR-92a inhibitors to the targeted cells. This is achieved by encapsulating miR-92a inhibitors in the core of the polyelectrolyte complex micelle while displaying targeting peptides against Vascular Cell Adhesion Molecule 1 (VCAM-1) on the surface. The expression of VCAM-1 increases in activated endothelium but remains low in healthy endothelium and VCAM-1-targeting drives active binding of nano-materials to inflamed endothelial cells.

### Example 3: Inhibition of miR-92a and Effects on Pathological Vascular Remodeling Under Disturbed Flow—Atherosclerosis

Introduction

A cohort of endothelial miRs have been shown to be differentially expressed between athero-susceptible arterial sites and athero-protected regions in both mouse and swine models as well as in humans. In particular, a pro-inflammatory micro RNA, miR-92a, is elevated in areas of athero-susceptibility and studies using cultured cells, animal models (mouse and swine), and human genetics (genome-wide association studies) have identified that increased presence of endothelial miR-92a is a major molecular signature of endothelial activation and pathological vascular remodeling under disturbed flow.

In the present study, the role of miR-92a as an underlying cause of the development of atherosclerotic lesions which also present disturbed flow conditions, was examined. The ability of the nanomedicine platform described in Example 1 to address this condition through the targeted delivery of oligonucleotides that inhibit miR-92a was examined.

Materials and Methods

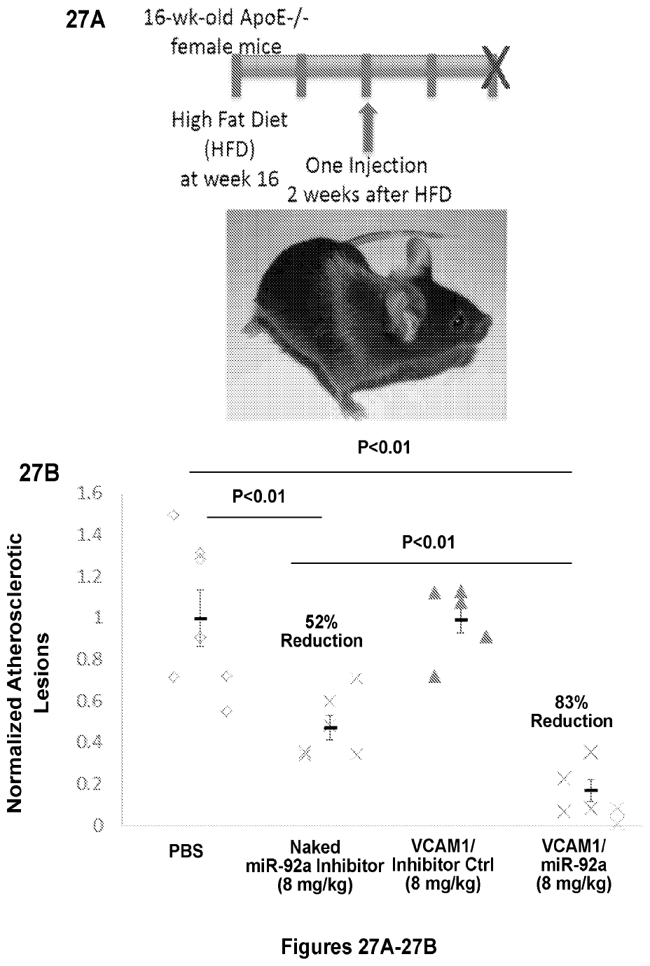

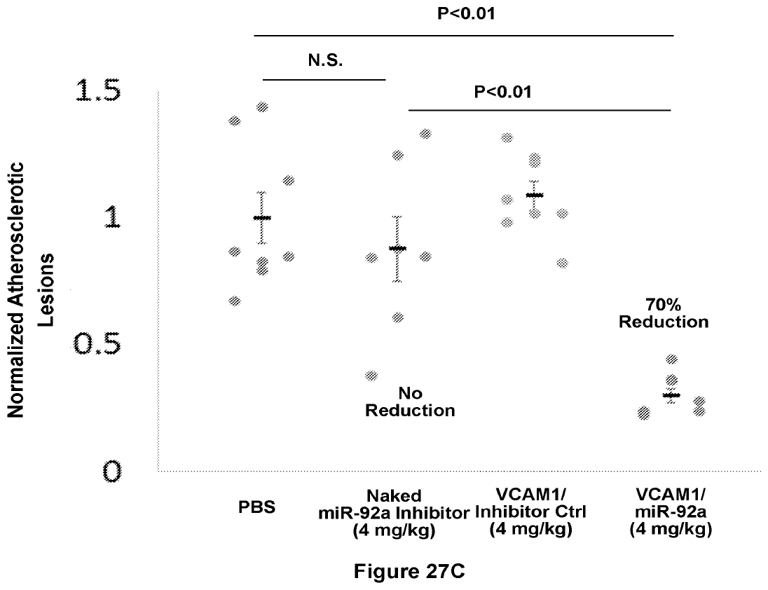

Atherosclerosis-prone apolipoprotein E-deficient (Apo E^{-/-}) mice display poor lipoprotein clearance with subsequent accumulation of cholesterol ester-enriched particles in the blood, which promote the development of atherosclerotic plaques. Therefore, the Apo E^{-/-} mouse model is well established for the study of human atherosclerosis and drug targets. Moreover, like humna lesions, atherosclerotic lesions in Apo E^{-/-} developed in arterial regions exposed to disturbed flow. Increased endothelial miR-92a by disturbed flow has been linked to atherosclerosis in humans and in Apo E^{-/-}. The study design of treating atherosclerosis in Apo E^{-/-} by targeted nanoparticles as described in Example 1 is shown in FIG. 27A.

Results

FIG. 27B shows a comparison between treatments administering the naked miR-92a inhibitor (8 mg/kg) and the micelle-encapsulated and -targeted miR-92a inhibitor (8 mg/kg), along with some controls. The statistically significant result in FIG. 27B is that naked miR-92a inhibitor produces a 55% reduction in lesion size, while targeting the miR-92a inhibitor to inflamed endothelium with a nanoparticle produces an 80% reduction. A follow-up series of experiments with the same protocol showed that, at a lower dosage of 4 mg/kg, the advantage of targeted nanoparticle delivery increased (80% reduction for targeted delivery vs 42% reduction for naked delivery) (data not shown).

Moreover, recent results demonstrated that when lower dosage of miR-92a inhibitor (4 mg/kg) was used, no reduction of the atherosclerosis was detected in Apo E^{-/-} mice treated with naked miR-92a inhibitor (4 mg/kg) but the atherosclerosis lesion was significantly reduced by 70% in mice treated with 4 mg/kg miR-92a inhibitor encapsulated in the VCAM1-targeting nanoparticles (FIG. 27C).

These results collectively demonstrate that the targeted nanoparticle approach significantly treat atherosclerotic lesions in vivo in a well-established mouse model of atherosclerosis. Therefore, such approaches may have clinically meaningful therapeutic effects in humans.

### Example 4: Inhibition of miR-92a and Effects on Pathological Vascular Remodeling Under Disturbed Flow—Restenosis

Introduction

The biology of restenosis and in-stent restenosis is similar to that of AVF failure both involving endothelial dysfunction, smooth muscle cell proliferation and matrix protein elaboration, and ultimately intimal hyperplasia. This similarity, and the preliminary success in the mouse model of AVF reported herein, suggests that the therapeutic nanoparticles described in Example 1 can also inhibit the restenosis that often occurs after angioplasty and insertion of a stent to relieve coronary artery blockage, which is a potentially larger indication than AVF. Over 1.8 million stents were implanted in the US in 2018, including drug eluting stents, and restenosis is anticipated to be in the range of 10-25% depending on the type of stent used. Thus, there exists a large unmet medical need for susceptible patients even when intervention with drug-eluting stents is used.

Materials and Methods

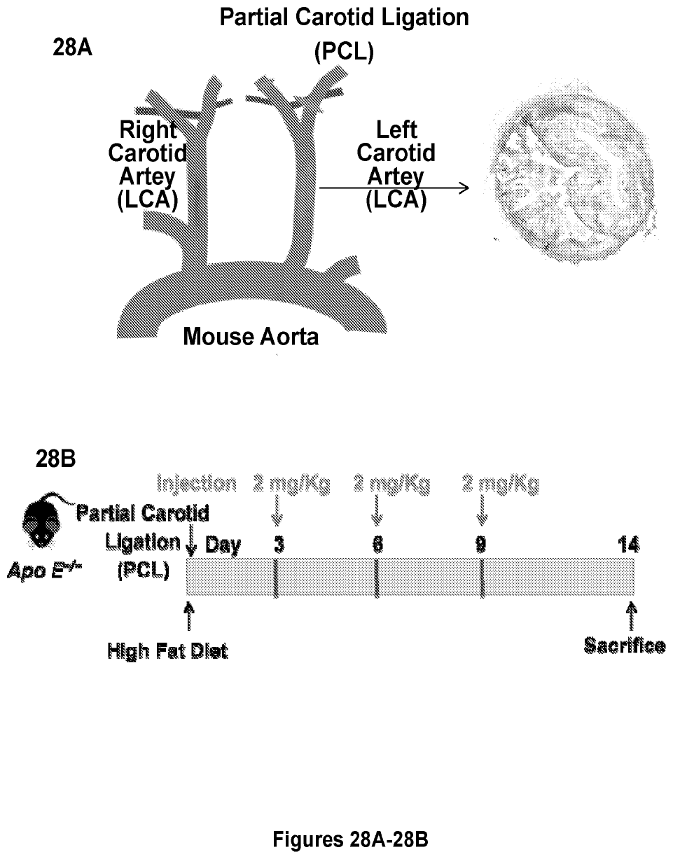

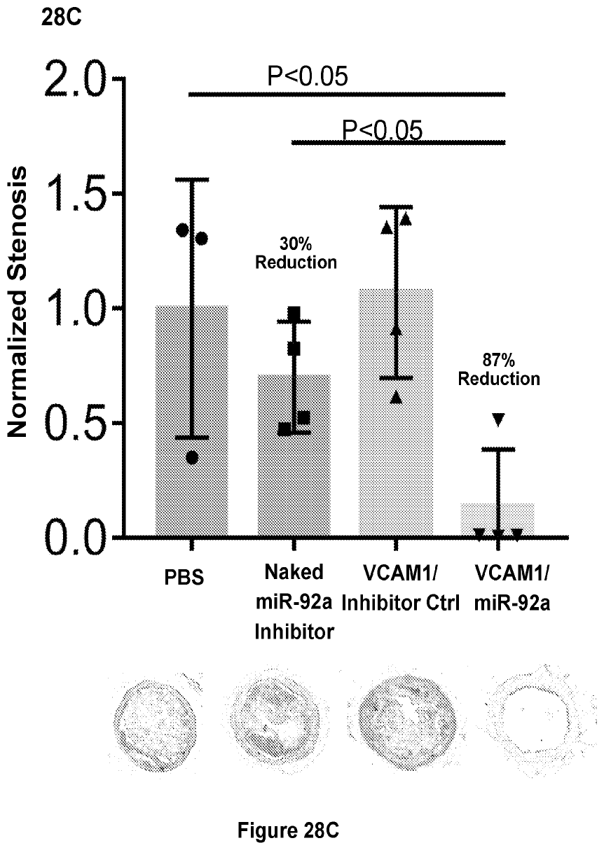

Carotid arterial stenosis can be induced in mice by a surgical procedure in the carotid arteries which introduces disturbed flow and increases miR-92a in endothelial cells. As shown in representative FIG. 28A, the left external carotid, internal carotid, and occipital artery were ligated with 6-0 silk suture while the superior thyroid artery remained intact: this partial carotid ligation results in flow disturbance in left carotid artery, leading to increased endothelial miR-92a expression and causing stenosis in two weeks in vivo. This mouse model has been widely-used to mimic the arterial stenosis in humans such as the in-stent restenosis. The treatment protocol is show in FIG. 28B. Specifically, three days after the partial carotid ligation, a tail vein injection was conducted of either naked miR-92a inhibitor or the miR-92a inhibitor encapsulated in a VCAM1-targeted micellar nanoparticle. Nanoparticles described in Example 1 were used. In both cases, a dose of 2 mg/kg of body weight was administered. Two more injections were conducted in day 6 and 9 after the partial carotid ligation. Mice were sacrificed 14 days after the partial carotid ligation to measure the stenosis in the partially-ligated left carotid arteries.

Results

The results (FIG. 28C) demonstrate that disturbed flow-induced stenosis in the partially-ligated carotid artery is significantly reduced by 87% by three injections of VCAM1-targeting nanoparticle encapsulating 2 mg/kg miR-92a inhibitors. However, injections of miR-92a inhibitors in the naked form had much less effect (30% reduction) on the disturbed flow-induced stenosis in mice.

Conclusion

The results demonstrated that the targeted nanoparticle platform significantly treats arterial stenosis in vivo.

The embodiments illustratively described herein suitably can be practiced in the absence of any element or elements, limitation or limitations that are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the embodiments claimed. Thus, it should be understood that although the present description has been specifically disclosed by embodiments, optional features, modification and variation of the concepts herein disclosed may be resorted to by those skilled in the art, and that such modifications and variations are considered to be within the scope of these embodiments as defined by the description and the appended claims. Although some aspects of the present disclosure can be identified herein as particularly advantageous, it is contemplated that the present disclosure is not limited to these particular aspects of the disclosure.

Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein.

Sequences.

| Sequence | SEQ ID NO: |
|---|---|
| REKA | 1 |
| VHPKQHR | 2 |
| NNQKIVNLK | 3 |
| EKVAQLEA | |
| DITWDQLWD | 4 |
| LMK | |
| CREKA | 5 |
| CGVHPKQHR | 6 |
| CGSPGWVRCG | 7 |

1. A targeted nanoparticle, comprising an inhibitor of microRNA-92a (miR-92a), wherein the targeted nanoparticle comprises a polyelectrolyte micelle and a targeting molecule.

2. (canceled)

3. The targeted nanoparticle of claim 1, wherein the polyelectrolyte micelle comprises a polyethylene glycol (PEG) domain and a domain of positively charged amino acids.

4. The targeted nanoparticle of claim 3, wherein the PEG domain comprises PEG having an average molecular weight of about 1,000 to about 100,000 Daltons.

5. The targeted nanoparticle of claim 4, wherein the domain of positively charged amino acids comprises repeats of lysine (K), arginine (R), and/or histidine (H).

6. The targeted nanoparticle of claim 5, wherein the domain of positively charged amino acids comprises repeats comprising about 2 to about 100 residues.

7. The targeted nanoparticle of claim 6, wherein the domain of positively charged amino acids comprises 30 repeats of lysine (K30).

8. The targeted nanoparticle of claim 1, wherein the targeting molecule comprises a peptide comprising the amino acid sequence REKA (SEQ ID NO: 1), VHPKQHR (SEQ ID NO: 2), NNQKIVNLKEKVAQLEA (SEQ ID NO: 3), DITWDQLWDLMK (SEQ ID NO: 4), CREKA (SEQ ID NO: 5), or CGVHPKQHR (SEQ ID NO: 6), CGSPGWVRCG (SEQ ID NO: 7).

9. The targeted nanoparticle of claim 8, wherein the targeted nanoparticle comprises VHPKQHR-PEG-K30, CGVHPKQHR-PEG-K30, NNQKIVNLKEKVAQLEA-PEG-K30, DITWDQLWDLMK-PEG-K30, REKA-PEG-K30, CREKA-PEG-K30, or CGSPGWVRCG-PEG-K30.

10. The targeted nanoparticle of claim 9, wherein the miR-92a inhibitor comprises hsa-miR-92a-3p.

11. The targeted nanoparticle of claim 10, wherein the miR-92a inhibitor comprises a concentration of about 2 μM.

12. A pharmaceutical composition, comprising:
a therapeutically effective amount of the targeted nanoparticle of claim 1; and
a pharmaceutically acceptable carrier, solvent, adjuvant, and/or diluent.

13. The pharmaceutical composition of claim 12 further comprising a secondary therapeutic agent.

14. The pharmaceutical composition of claim 13, wherein the secondary therapeutic agent comprises one or more of an anticoagulant, an antiplatelet agent, an angiotensin-converting enzyme inhibitor, an angiotensin II receptor blocker, an angiotensin-receptor neprilysin inhibitor, a beta blocker, a calcium channel blocker, a cholesterol-lowering medication, a digitalis preparation, a diuretic, a vasodilator, an anti-inflammatory medication, an IL-1b blocker, an inflammasome blocker, dehydroepiandrosterone sulfate, a myeloperoxidase inhibitor, a dipeptidyl peptidase-4 (DPP-4) inhibitor, a nitric oxide synthase activator, and/or a small GTPAse RhoA inhibitor.

15. The pharmaceutical composition of claim 12, wherein the pharmaceutical composition is formulated for oral, intravenous, topical, ocular, buccal, systemic, nasal, injection, transdermal, rectal, or vaginal administration.

16. The pharmaceutical composition of claim 12, wherein the pharmaceutical composition is formulated for inhalation or insufflation.

17-27. (canceled)

28. A method of treating a vascular disorder in a subject, comprising:
administering to the subject a therapeutically effective amount of a pharmaceutical composition comprising a targeted nanoparticle comprising an miR-92a inhibitor, wherein the targeted nanoparticle is preferentially targeted to inflamed endothelial cells associated with the vascular disorder; and
reducing inflammation at the site of the inflamed endothelial cells.

29. The method of claim 28, wherein the vascular disorder comprises one or more of arteriovenous fistula (AVF) failure, stenosis, restenosis, and atherosclerosis.

30. The method of claim 29, wherein the method results in one or more of greater lumen cross-sectional area, greater lumen diameter, or increased flow rate compared to a control at the site of the inflamed endothelial cells.

31. A method of promoting endothelial wound healing in a subject, comprising:
administering to the subject a therapeutically effective amount of a pharmaceutical composition comprising a targeted nanoparticle comprising an miR-92a inhibitor, wherein the targeted nanoparticle is preferentially targeted to inflamed endothelial cells associated with the endothelial wound; and
reducing inflammation at the site of the inflamed endothelial cells.

32. The method of claim 31 further comprising stimulating endothelial growth at the site of the wound.
